(19) United States
(12) Patent Application Publication
(10) Pub. No.: US 2018/0293360 A1
(43) Pub. Date: Oct. 11, 2018

(54) SYSTEMS AND METHODS FOR PREDICTING VITREAL HALF-LIFE OF THERAPEUTIC AGENT-POLYMER CONJUGATES

(71) Applicant: Genentech, Inc., South San Francisco, CA (US)

(72) Inventors: Robert Kelley, South San Francisco, CA (US); Justin Scheer, South San Francisco, CA (US); Whitney Shatz, South San Francisco, CA (US)

(21) Appl. No.: 15/766,319

(22) PCT Filed: Oct. 5, 2016
(86) PCT No.: PCT/US2016/055421

§ 371 Date: Apr. 5, 2018

Related U.S. Application Data
(60) Provisional application No. 62/238,393, filed on Oct. 7, 2015.

(51) Int. Cl. G06F 19/00; G06F 17/18

(52) U.S. Cl. CPC G06F 19/704; A61K 47/61; G06F 17/18

(57) ABSTRACT

Disclosed are systems and methods for estimating the vitreal half-life of a therapeutic agent. In particular, systems and methods are disclosed for predicting the vitreal half-life of a therapeutic agent conjugated to a polymer that make use of an empirically-derived relationship of vitreal half-life to the hydrodynamic radius of a candidate therapeutic agent-polymer conjugate. The present disclosure is further directed to the use of the systems and methods disclosed herein to design a candidate therapeutic agent-polymer conjugate with a preselected vitreal half-life.

### RELATED APPLICATIONS

This application claims the benefit of U.S. Provisional Application No. 62/238,393, entitled “SYSTEMS AND METHODS FOR PREDICTING VITREAL HALF-LIFE OF THERAPEUTIC AGENT-POLYMER CONJUGATES” and filed Oct. 7, 2015, which is hereby incorporated by reference herein in its entirety.

### FIELD OF THE DISCLOSURE

The present disclosure relates generally to systems and methods for predicting the vitreal half-life of a therapeutic agent-polymer conjugate. In particular, the present disclosure relates to systems and methods for predicting the vitreal half-life of a therapeutic agent conjugated to a polymer by an empirically-derived relationship between vitreal half-life and the hydrodynamic radius of the therapeutic agent-polymer conjugate. The present disclosure is further directed to the use of the systems and methods disclosed herein to design a therapeutic agent-polymer conjugate with a predetermined vitreal half-life.

### BACKGROUND

The administration of therapeutic agents for the treatment of many internal eye disorders, such as retinal disorders, are challenging due to the relatively isolated location of these structures, as well as the relatively short residence time of the therapeutic agents typically used for these ocular treatments within the eye. As a result, maximum benefit to the patient is typically obtained through frequent dosing by intravitreal injections of the therapeutic agents. However, increased patient convenience and compliance, as well as decreased risk of inflammation, may be more easily obtainable using treatments that require less frequent dosing.

To achieve positive clinical outcomes with less frequent dosing, a variety of technologies have been considered to achieve long acting delivery (LAD) of therapeutic agents. Existing technologies for long acting delivery (LAD) include slow release formulations of the therapeutic agent, molecular modification of the therapeutic agent to extend the therapeutic agent's half-life, and administration of the therapeutic agent using implantable devices. Typically, these approaches require in vivo pre-clinical testing in an animal model, such as a rabbit, to assess feasibility for sustained delivery. The existing paradigm for assessing the vitreal half-life of the therapeutic agent within the eye includes initial testing of intravitreal dosing in rabbits to assess the vitreal half-life of the candidate therapeutic agent. However, this existing paradigm may not be as well-suited for the evaluation of certain classes of candidate therapeutic agents, such as human or humanized antibodies. In vivo testing of vitreal half-life using an animal model is time-consuming and expensive. In addition, the results of the animal model may be unreliable due to immune responses to the therapeutic agent over prolonged exposure times. Typically, most human or humanized antibodies are immunogenic in rabbits, a problem that is exacerbated over an extended duration of exposure. Immune reactions of the animal model to the candidate therapeutic agent under consideration confounds interpretation of pharmacokinetic (PK) data obtained during initial testing and may result in flawed conclusions about the potential of a therapeutic agent-polymer conjugate if a human or humanized antibody is used as the therapeutic agent for these feasibility studies in rabbits.

Accordingly, there exists a need for systems and methods of predicting the vitreal half-life of candidate therapeutic agents in various formulations, including therapeutic agent-polymer conjugates that make use of simple in vitro measurements to enable relatively rapid and inexpensive screening of a candidate formulation of a therapeutic agent. In addition, a need exists for systems and methods of predicting the vitreal half-life of candidate therapeutic agents in various formulations that are compatible with a wide variety of therapeutic agents including human or humanized antibodies. There also exists a need for systems and methods for designing a formulation of a therapeutic agent-polymer conjugate that achieves a preselected vitreal half-life upon administration by intravitreal injection.

### SUMMARY OF THE DISCLOSURE

In one aspect, the present disclosure is directed to a method for identifying a therapeutic agent-polymer conjugate having a preselected vitreal half-life. In this aspect, the method comprises: a) determining a hydrodynamic radius (RH) of the therapeutic agent-polymer conjugate; b) transforming the RH to a predicted vitreal half-life of the therapeutic agent-polymer conjugate according to a predetermined vitreal half-life-RH relation; and c) assessing whether the predicted vitreal half-life is greater than or equal to the preselected vitreal half-life. The method may further comprise: d) modifying the polymer moiety of the therapeutic agent-polymer conjugate to increase the RH if the predicted vitreal half-life is less than the preselected vitreal half-life, and repeating a)-c) until the predicted vitreal half-life of the conjugate is greater than or equal to the preselected vitreal half-life. The method may additionally comprise: e) selecting the therapeutic agent-polymer conjugate from c) wherein the predicted vitreal half-life of the conjugate is greater than or equal to the preselected vitreal half-life. The method may additionally comprise: f) determining an in vivo vitreal half-life of the therapeutic agent-polymer conjugate from c) using an animal model.

In another aspect, the present disclosure is directed to a method for selecting a therapeutic agent-polymer conjugate having a predicted vitreal half-life that is greater than or equal to a preselected vitreal half-life for use in an ocular therapy. In this aspect, the method comprises: a) preparing a plurality of candidate therapeutic agent-polymer conjugates, wherein each candidate therapeutic agent-polymer conjugate of the plurality comprises the therapeutic agent and a polymer moiety, and further wherein each polymer moiety has a different composition than each other polymer moiety in the plurality. The method further comprises: b) determining a hydrodynamic radius (RH) for each therapeutic agent-polymer conjugate of the plurality; c) transforming each RH to a predicted vitreal half-life for each therapeutic agent-polymer conjugate of the plurality according to a predetermined vitreal half-life-RH relation; d) assessing whether each predicted vitreal half-life is greater than or equal to the preselected vitreal half-life; and e) selecting one of the candidate therapeutic agent-polymer conjugate with a predicted vitreal half-life that is greater than or equal to the preselected vitreal half-life for the ocular therapy.

In yet another additional aspect, the present disclosure is directed to a method for identifying a therapeutic agent-polymer conjugate having a preselected vitreal half-life. In this aspect, the method is implemented by a computing device including at least one processor in communication with a memory. The method comprises: a) receiving, by the computing device, a hydrodynamic radius (RH) of the therapeutic agent-polymer conjugate; b) transforming, by the computing device, the RH to a predicted vitreal half-life of the therapeutic agent-polymer conjugate according to a predetermined vitreal half-life-RH relation; c) assessing whether the predicted vitreal half-life is greater than or equal to the preselected vitreal half-life; and d) displaying, by the computing device, on a user interface of the computing device, the predicted vitreal half-life. The method may further comprise: d) displaying, by the computing device, on a user interface of the computing device, the therapeutic agent-polymer conjugate comprising the therapeutic agent and the modified polymer moiety, and the predicted vitreal half-life; and e) modifying the polymer moiety of the therapeutic agent-polymer conjugate to increase the RH if the predicted vitreal half-life is less than the preselected vitreal half-life, and repeating a)-d) until the predicted vitreal half-life of the conjugate is greater than or equal to the preselected vitreal half-life.

In yet another aspect, the present disclosure is directed to a computing device that comprises at least one processor in communication with a memory, wherein the at least one processor is programmed to: a) receive a hydrodynamic radius (RH) of the therapeutic agent-polymer conjugate; b) transform the RH to a predicted vitreal half-life of the therapeutic agent-polymer conjugate according to a predetermined vitreal half-life-RH relation; c) assess whether the predicted vitreal half-life is greater than or equal to the preselected vitreal half-life; and d) display, on a user interface of the computing device, the therapeutic agent-polymer conjugate comprising the therapeutic agent and the modified polymer moiety, and the predicted vitreal half-life. The at least one processor may be further programmed to: e) modify the polymer moiety of the therapeutic agent-polymer conjugate to increase the RH if the predicted vitreal half-life is less than the preselected vitreal half-life, and repeat a)-d) until the predicted vitreal half-life of the conjugate is greater than or equal to the preselected vitreal half-life. In this aspect, the polymer moiety may be modified by the computing device.

In yet another aspect, the present disclosure is directed to a computer-readable storage medium having computer-executable instructions embodied thereon, wherein when executed by a computing device including at least one processor in communication with a memory, the computer-executable instructions cause the computing device to: a) receive a hydrodynamic radius (RH) of the therapeutic agent-polymer conjugate; b) transform the RH to a predicted vitreal half-life of the therapeutic agent-polymer conjugate according to a predetermined vitreal half-life-RH relation; c) assess whether the predicted vitreal half-life is greater than or equal to the preselected vitreal half-life; and d) display, on a user interface of the computing device, the therapeutic agent-polymer conjugate comprising the therapeutic agent and the modified polymer moiety, and the predicted vitreal half-life. The computer-executable instructions may further cause the computing device to: e) modify the polymer moiety of the therapeutic agent-polymer conjugate to increase the RH if the predicted vitreal half-life is less than the preselected vitreal half-life, and repeat a)-d) until the predicted vitreal half-life of the conjugate is greater than or equal to the preselected vitreal half-life. The computer-executable instructions may also cause the computing device to modify the polymer moiety by providing one or more suggested polymers from a database of polymers to be evaluated for use as polymer moieties in the therapeutic agent-polymer conjugate.

In yet another aspect, the present disclosure relates to selection of a therapeutic agent-polymer conjugate having a desired vitreal half-life and packaging a dosage thereof in a storage device suitable for use in the administration thereof to a patient. In one particular aspect, the storage device is a pre-filled syringe or alternatively an ampule or vial configured to permit the withdrawal of at least one dosage via the syringe.

In yet another aspect, the present disclosure relates to a system for identifying a therapeutic agent-polymer conjugate having a preselected vitreal half-life using a computing device comprising at least one processor in communication with a memory, the memory comprising a plurality of modules, each module comprising instructions configured to execute using the at least one processor. The plurality of modules includes: a first module to receive a hydrodynamic radius (RH) of the therapeutic agent-polymer conjugate; a second module to transform the RH to a predicted vitreal half-life of the therapeutic agent-polymer conjugate according to a predetermined vitreal half-life-RH relation; a third module to assess whether the predicted vitreal half-life is at least the preselected vitreal half-life; and a fourth module to display, on a user interface of the computing device, the therapeutic agent-polymer conjugate comprising the therapeutic agent and the modified polymer moiety, and the predicted vitreal half-life. The plurality of modules further include a fifth module to modify the polymer moiety of the therapeutic agent-polymer conjugate to increase the RH if the predicted vitreal half-life is less than the preselected vitreal half-life, and to re-execute the instructions of the first, second, third, and fourth modules until the predicted vitreal half-life of the conjugate is greater than or equal to the preselected vitreal half-life.

### BRIEF DESCRIPTION OF THE DRAWINGS

FIG. 1 is a graph depicting the vitreal half-lives of several therapeutic agents, therapeutic agent-polymer conjugates, and surrogate-polymer conjugates after intravitreal injection within a rabbit eye as a function of hydrodynamic radius.

FIG. 2 is a graph depicting the vitreal half-lives of several therapeutic agents, therapeutic agent-polymer conjugates, and surrogate-polymer conjugates after intravitreal injection within a rabbit eye as a function of molecular weight.

FIG. 3 is a graph depicting the vitreal half-lives of several therapeutic agents, therapeutic agent-polymer conjugates, and surrogate-polymer conjugates after intravitreal injection within a rabbit eye as a function of hydrodynamic radius.

FIG. 4 is a flow chart illustrating a method of identifying a therapeutic agent-polymer conjugate with a preselected vitreal half-life in one embodiment.

FIG. 5 is a graph depicting the vitreal half-lives of several therapeutic agents and therapeutic agent-polymer conjugates after intravitreal injection within a rabbit eye and within a monkey eye as a function of hydrodynamic radius.

FIG. 6 is a flow chart illustrating a method of designing a therapeutic agent-polymer conjugate with greater than or equal to a preselected vitreal half-life.

FIG. 7A depicts an exemplary therapeutic agent chemical structure conjugated to a single polymer moiety. FIG. 7B depicts an exemplary therapeutic agent chemical structure of two therapeutic agents conjugated to a single polymer moiety. FIG. 7C depicts an exemplary therapeutic agent chemical structure of multiple therapeutic agents conjugated to multiple arms of a branched polymer moiety.

FIG. 8 is a block diagram illustrating a server system.

FIG. 9 is a block diagram illustrating a computing device.

FIG. 10 is a graph depicting elimination of a surrogate from the eye of a rabbit model after an intravitreal injection.

FIG. 11 is a graph depicting multiple eliminations of a surrogate from the eye of a rabbit model after repeated intravitreal injections.

FIG. 12A is a graph depicting a correlation function used to measure RH using quasi elastic light scattering (QELS). FIG. 12B is a graph depicting a representative QELS signal.

FIG. 13 is a graph depicting the vitreal half-life of several surrogate-polymer conjugates from a rabbit model after an intravitreal injection as a function of hydrodynamic radius.

FIG. 14 is a graph depicting elimination of several surrogate-polymer conjugates from the eye of a rabbit model after intravitreal injection.

FIG. 15A is a graph depicting the effect of FcRn binding on the vitreal half-life of a therapeutic agent. FIG. 15B is a graph depicting the effect of FcRn binding on the systemic half-life of a therapeutic agent.

FIG. 16 is a graph depicting the effect of net charge of a therapeutic agent on the corresponding vitreal half-lives.

The following detailed description of the embodiments of the disclosure refers to the accompanying drawings. The same reference numbers in different drawings may identify the same or similar elements. Also, the following detailed description does not limit the claims.

### DETAILED DESCRIPTION

As further detailed herein, the present disclosure is directed to systems and methods derived from the discovery that the vitreal half-life of a therapeutic agent-polymer conjugate comprising a therapeutic agent and a polymer as described herein below may be accurately predicted based on the hydrodynamic radius (RH) of the therapeutic agent-polymer conjugate.

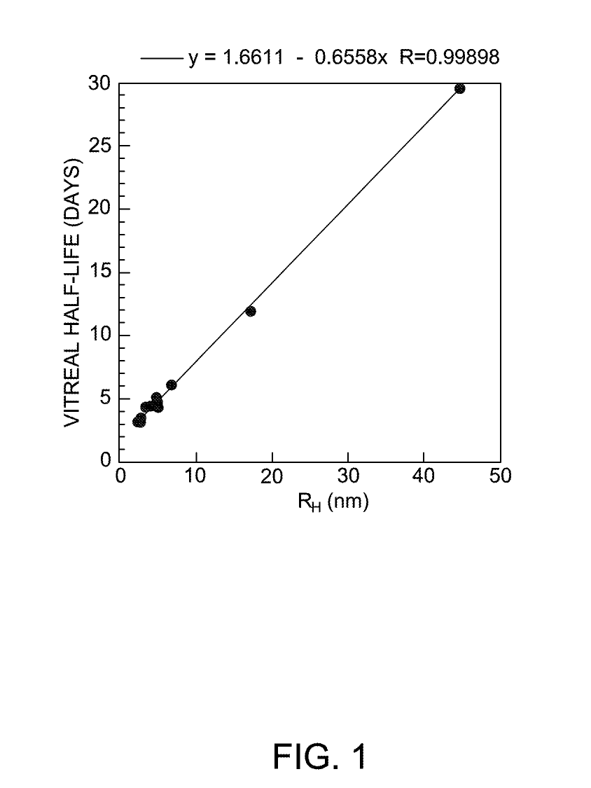

The systems and methods described herein facilitate the identification of a suitable therapeutic agent-polymer conjugate for use in an ocular therapy. The systems and methods disclosed herein enable an ocular therapeutic method that is enhanced by selection of a suitable, e.g., a relatively high, vitreal half-life. In various embodiments, the method makes use of a predetermined vitreal half-life-RH relation that is empirically derived by correlating the vitreal half-lives with the hydrodynamic radii and/or hydrodynamic volumes previously measured for a plurality of therapeutic agent-polymer conjugates. FIG. 1 is a graph depicting the predetermined vitreal half-life-RH relation in one embodiment, in which the predetermined vitreal half-life-RH relation is empirically derived using measurements of RH and measurements of vitreal half-life in a rabbit or other animal model as described herein.

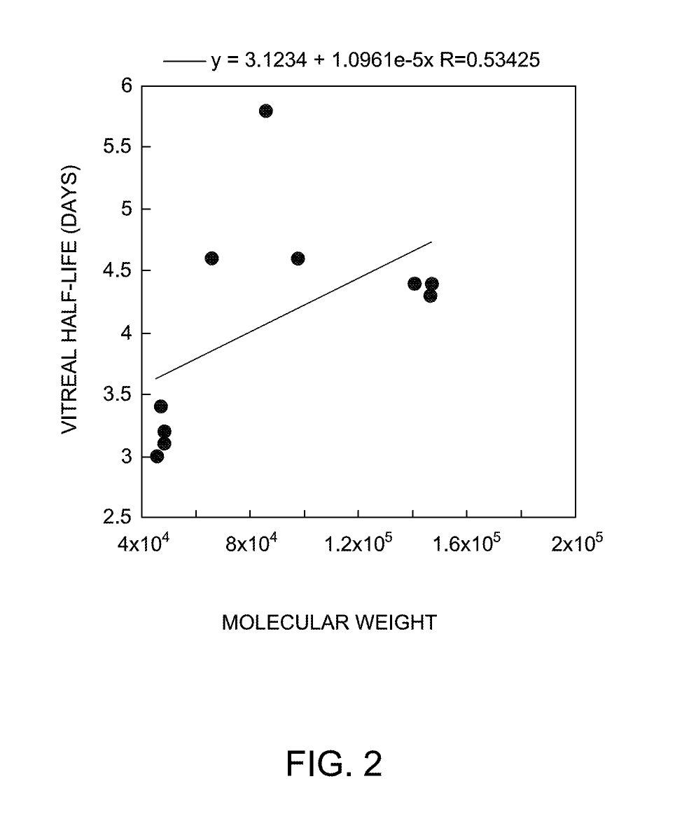

Without being limited to any particular theory, the relatively high degree of correlation of vitreal half-life with respect to hydrodynamic radius and/or hydrodynamic volume derives from the discovery that the clearance of therapeutic agent from the vitreous humor may be dominated by diffusive processes, rather than by convective and/or filtration processes typical of systemic clearance processes. By way of non-limiting example, it has been demonstrated that systemic half-lives of therapeutic agent-polymer conjugates do not exhibit a linear relationship with respect to hydrodynamic radius (see Koumenis et al. 2000 Int. J. Pharm. 198:83-95). As described in detail herein below, the vitreal half-lives of the therapeutic agent-polymer conjugates exhibit a linear correlation with respect to hydrodynamic radius (RH). As used herein, the “hydrodynamic radius (RH)” of a compound refers to the radius of a hard sphere that diffuses at the same rate as the compound in solution. As such, vitreal half-life, which is highly dependent upon diffusive processes, correlates well with RH, which quantifies the diffusive behavior of a therapeutic agent, a polymer, and/or a therapeutic agent-polymer conjugate. By contrast, as illustrated in FIG. 2, the vitreal half-lives of the same therapeutic agents and therapeutic agent-polymer conjugates illustrated in FIG. 1 (in particular the PEG-Fab conjugates) correlate poorly with their corresponding molecular weights (see also Missell 2012 Pharm. Res. 29:3251-3272).

The predetermined vitreal half-life-RH relation derived from a correlation of measured vitreal half-lives and corresponding hydrodynamic radii for a plurality of therapeutic agents and/or therapeutic agent-polymer conjugates with a range of hydrodynamic radii enables the accurate transformation of hydrodynamic radii, a quantity that may be readily measured using existing methods, to a vitreal half-life, which previously required onerous and time-consuming in vivo measurements using animal models. This predetermined vitreal half-life-RH relation is included in the systems and methods for identifying a therapeutic agent-polymer conjugate having a preselected vitreal half-life, in order to accurately predict the vitreal half-life of a variety of therapeutic agent-polymer conjugates more rapidly and at lower cost compared to existing in vivo screening methods.

### A. Definitions

Unless otherwise defined, all terms of art, notations, and other scientific terminology used herein are intended to have the ordinary meanings commonly understood by those of ordinary skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, “therapeutic agent” refers to any substance or combination of substances used in a finished pharmaceutical product (FPP), intended to furnish pharmacological activity or to otherwise have direct effect in the diagnosis, cure, mitigation, treatment or prevention of disease, or to have direct effect in restoring, correcting, or modifying physiological function when administered to a patient. Non-limiting examples of therapeutic agents include antibodies and fragments thereof, proteins and fragments thereof, and small molecules.

As used herein, “surrogate” or “surrogate compound” refer to a compound used to evaluate an aspect of a formulation of a therapeutic agent with a similar structure. One non-limiting example of a surrogate is rabFab, a rabbit antibody used to evaluate ocular PK characteristics as described in Examples 1-3 below.

As used herein, the term “preselected vitreal half-life” refers to a vitreal half-life selected by a user of the systems and methods of this disclosure that represents a desired or targeted level of vitreal half-life for the therapeutic agent-polymer conjugate. The predicted vitreal half-life may be compared to the preselected vitreal half-life to determine whether a therapeutic agent-polymer conjugate may be identified as suitable for use as an ocular therapy.

As used herein, the term “predicted vitreal half-life” refers to a vitreal half-life that is predicted for a therapeutic agent-polymer conjugate by the systems and methods of this disclosure. Typically, the predicted vitreal half-life is produced by the transformation of a user-supplied hydrodynamic radius (RH) according to a predetermined vitreal half-life-RH relation.

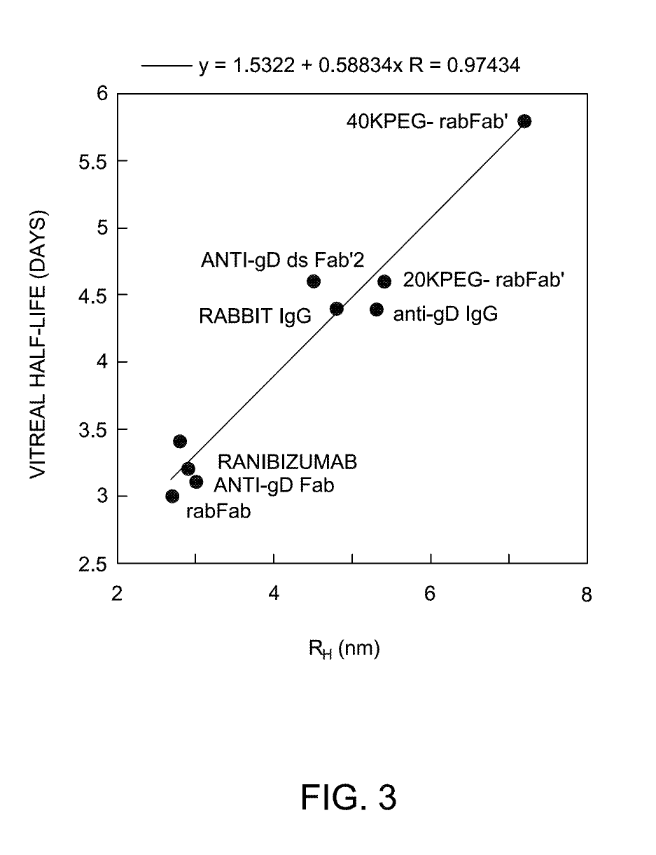

As used herein, the term “predetermined vitreal half-life-RH relation” refers to an equation specifying a correlation between the vitreal half-life and the hydrodynamic radius (RH) of a therapeutic agent-polymer conjugate used to transform the RH supplied by the user for the therapeutic agent-polymer conjugate to the predicted vitreal half-life according to the systems and methods of this disclosure. By way of non-limiting example, the predetermined vitreal half-life-RH relation may be a linear regression equation obtained using a linear regression analysis of a dataset comprising measured vitreal half-lives of a plurality of therapeutic agent-polymer conjugates and the corresponding measured RH values, as illustrated in FIG. 3.

As used herein, the “hydrodynamic radius (RH)” of a compound refers to the radius of a hard sphere that diffuses at the same rate as the compound in solution. The “hydrodynamic radius” of a therapeutic agent-polymer conjugate can vary depending on the polymer's molecular weight, the polymer's chemical structure (linear, branched, multi-armed, etc.), as well as how well the polymer interacts with the solvent.

As used herein, the “hydrodynamic volume” refers to the volume a polymer coil or therapeutic agent-polymer conjugate occupies when it is in solution. The “hydrodynamic volume” of a polymer or therapeutic agent-polymer conjugate can vary depending on its molecular weight and how well it interacts with the solvent. For example, every ethylene oxide repeating unit of PEG is known to bind 2-3 water molecules. Hydrodynamic volume may be measured in units of molecular radius.

The term “charged molecule” or “charged moiety” as used herein, refers to any moiety or molecule possessing a formal charge. The charged molecule may be permanently charged by virtue of its inherent structure, or as a result of its covalent bonding to another atom. The charged molecule may also possess a formal charge by virtue of the pH conditions existing of the surrounding environment, such as for example, the environment existing during drug delivery. The charge on the molecule may be either positive (cationic) or negative (anionic). The charged molecule can comprise positive charges or negative charges only. The charged molecule can also comprise a combination of both positive and negative charges. In a particular embodiment, the charged molecule has a net anionic charge. Chemical groups that impart a positive charge to a charged molecule include, but are not limited to, ionizable nitrogen atoms, such as in amino-containing compounds. Chemical groups that impart a negative charge to a charged molecule include, but are not limited to, carboxylate, sulfate, sulfonate, phosphonate or phosphate groups.

A charged molecule or a biologically active molecule-charged molecule conjugate are optionally accompanied by one or more “counterions”. Counterions accompanying a charged molecule or a biologically active molecule-charged molecule conjugate may be considered to be part of the charged molecule. Counterions for both the charged molecule and the resulting biologically active molecule-charged molecule conjugate may result in pharmaceutically acceptable salts. Suitable anionic counterions include, but are not limited to, chloride, bromide, iodide, acetate, methanesulfonate, succinate, and the like. Suitable cationic counterions include, but are not limited to, Na⁺, K⁺, Mg²⁺, Ca²⁺, NH⁴⁺ and organic amine cations. Organic amine cations include, but are not limited to, tetraalkylammonium cations and organic amines, that together with a proton, form a quaternary ammonium cations. Examples of organic amines capable of forming quaternary ammonium cations include, but are not limited to, mono- and di-organic amines, mono- and di-amino acids and mono- and di-amino acid esters, diethanolamine, ethylene diamine, methylamine, ethylamine, diethylamine, triethylamine, glucamine, N-methylglucamine, 2-(4-imidazolyl) ethyl amine), glucosamine, histidine, lysine, arginine, tryptophan, piperazine, piperidine, tromethamine, 6′-methoxy-cinchonan-9-ol, cinchonan-9-ol, pyrazole, pyridine, tetracycline, imidazole, adenosine, verapamil and morpholine.

The term “polymer” refers to any large molecule, or macromolecule, composed of many repeated subunits, or monomers. The molecular weight of the polymer is typically at least about 20,000 Da.

The term “monomer” refers to a small molecule that is a repeat unit within a polymer. A plurality of monomers is covalently bonded to form a polymer.

The term “linear polymer” refers to a polymer characterized by a single linear chain of monomers in which each monomer is joined end-to-end with the adjacent monomers.

A “branched polymer” refers to a polymer characterized by a main monomer chain and at least one substituent side chains.

The term “multi-armed polymer” refers to a branched polymer characterized by at least two relatively long substituent side chains referred to herein as “arms”.

The term “therapeutic agent-polymer conjugate” refers to a composition comprising at least one therapeutic agent molecule covalently attached to a polymer. In the context of the “therapeutic agent-polymer conjugate”, the polymer is referred to herein as a “polymer moiety”. Typically, the therapeutic agent is covalently attached to the polymer moiety in a manner that minimizes impact on the activity of the therapeutic agent.

The term “copolymer” refers to a polymer made from more than one kind of monomer. A copolymer may comprise one of several configurations, including block (e.g., AAAAAAABBBBBBB), random (e.g., AABAABBABBBBAA), or repeating configurations (e.g., ABABABABABAB).

The term “covalent bond” refers to the joining of two atoms that occurs when they share a pair of electrons.

The term “non-peptidic polymer”, as used herein, refers to an oligomer substantially without amino acid residues.

The term “non-nucleic acid polymer”, as used herein, refers to an oligomer substantially without nucleotide residues.

“Ocular delivery” and “ophthalmic delivery” refer to delivery of a compound, such as a biologically active molecule, to an eye tissue or fluid. “Ocular iontophoresis” refers to iontophoretic delivery to an eye tissue or fluid. Any eye tissue or fluid can be treated using iontophoresis. Eye tissues and fluids include, for example, those in, on or around the eye, such as the vitreous, conjunctiva, cornea, sclera, iris, crystalline lens, ciliary body, choroid, retina and optic nerve.

The term “nonproteinaceous polymer” typically refers to a hydrophilic synthetic polymer, i.e., a polymer not otherwise found in nature. Non-limiting examples of suitable nonproteinaceous polymers include polyvinyl alcohol; polyvinylpyrrolidone; polyalkylene ethers such as polyethylene glycol (PEG); polyoxyalkylenes such as polyoxyethylene, polyoxypropylene, and block copolymers of polyoxyethylene and polyoxypropylene (Pluronics); polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g., polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g., hyaluronic acid; polymers of sugar alcohols such as polysorbitol and polymannitol; and heparin or heparosan.

The term “polyethylene glycol,” or “PEG” refers to any polymer of general formula H(OCH₂CH₂)ₙOH, wherein n is greater than 3. In one embodiment, n is from about 4 to about 4000. In another embodiment, n is from about 20 to about 2000. In one embodiment, n is about 450. In one embodiment, PEG has a molecular weight of from about 800 Daltons (Da) to about 100,000 Da. In further embodiments, the polyethylene glycol is a 20 kDa PEG, 40 kDa PEG, or 80 kDa PEG. The average relative molecular mass of a polyethylene glycol is sometimes indicated by a suffixed number. For example, a PEG having a molecular weight of 4000 Daltons (Da) may be referred to as “polyethylene glycol 4000”). A PEG-conjugated product may be referred to as a PEGylated product.

As used herein, an element or step recited in the singular and proceeded with the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

### B. Method of Identifying Therapeutic Agent-Polymer Conjugate with Preselected Vitreal Half-Life

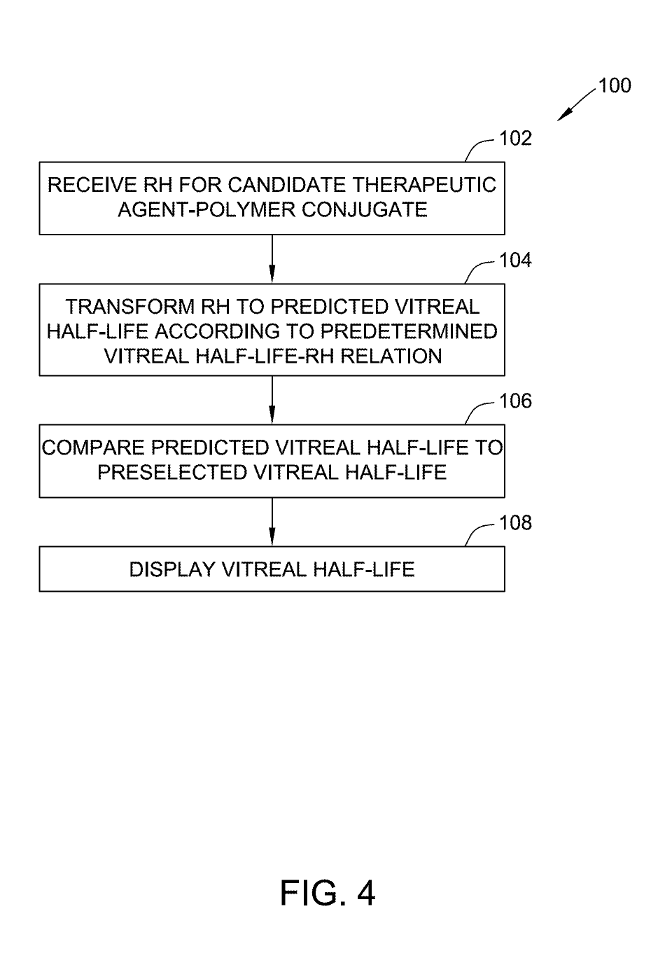

FIG. 4 is a flow chart illustrating the steps of one embodiment of a method 100 of the present invention for identifying a therapeutic agent-polymer conjugate having a preselected vitreal half-life. In various embodiments, the method 100 is implemented by a computing device including at least one processor in communication with a memory as described in further detail herein below.

Receive Hydrodynamic Radius

Referring again to FIG. 4, the method 100 includes receiving a hydrodynamic radius (RH) of the therapeutic agent-polymer conjugate at step 102. In various aspects, the RH of the therapeutic agent-polymer conjugate may be obtained by any known method including, but not limited to, empirically measuring the RH from a sample of the therapeutic agent-polymer conjugate; estimating RH from a chemical structure of the therapeutic agent-polymer conjugate using any known method (including, for example, estimating RH for the therapeutic agent-polymer conjugate using a known RH value for a conjugate having a substantially similar chemical structure); and retrieving a previously published RH value for the therapeutic agent-polymer conjugate. Non-limiting examples of suitable methods for empirically measuring the RH of the therapeutic agent-polymer conjugate include: quasi elastic light scattering (QELS), fluorescence correlation spectroscopy (FCS), pulse field NMR, and UV area imaging. In one embodiment, the RH value for the therapeutic agent-polymer conjugate is measured using quasi elastic light scattering (QELS). An example of the measurement of RH using the QELS method is provided in the Examples herein below (see also Roche et al. (1993) Biochemistry 32:5629).

In some embodiments, the RH of the therapeutic agent-polymer conjugate may be received by the computing device via an input device provided for receiving input from a user including, but not limited to, a keyboard, a touch sensitive panel, and the like. In other embodiments, the RH of the therapeutic agent-polymer conjugate may be received by the computing device via a communications interface that operatively couples the computing device to a device used for measuring RH including, but not limited to, a QELS module.

Transform RH to Predicted Vitreal Half-Life

Referring again to FIG. 4, the method 100 may further include transforming the received RH to a predicted vitreal half-life according to a predetermined vitreal half-life-RH relation at step 104. As discussed herein previously, the predetermined vitreal half-life-RH relation may be obtained empirically by correlating a plurality of vitreal half-lives measured for a plurality of therapeutic agent-polymer conjugates with the corresponding hydrodynamic radii (RH) measured for the plurality of therapeutic agent-polymer conjugates. Without being limited to any particular theory, the predetermined vitreal half-life-RH relation is characterized by a relatively high degree of correlation between the vitreal half-life and the hydrodynamic radius for therapeutic agent-polymer conjugates that include structurally diverse polymer moieties.

By way of non-limiting example, the therapeutic agent-polymer conjugates referenced in FIG. 1 include a variety of polymer moieties that include linear polymers (PEG and hyaluronic acid (HA)) as well as therapeutic agents lacking any polymer moieties, yet the predetermined vitreal half-life-RH relation is characterized as a well-correlated linear regression for all therapeutic agent-polymer conjugates included in FIG. 1.

In various embodiments, any known suitable correlation method may be used to obtain the predetermined vitreal half-life-RH relation without limitation. Non-limiting examples of correlation methods suitable for obtaining the predetermined vitreal half-life-RH relation include: linear regression methods, nonlinear regression methods, polynomial curve fitting, curve fitting to other functions such as trigonometric functions or logarithmic functions, and any other suitable correlation method. In one particular embodiment, the predetermined vitreal half-life-RH relation may be expressed in an equation form comprising the dependent variable vitreal half-life as a function of the independent variable RH. In this embodiment, the predetermined vitreal half-life-RH relation may be stored within a memory of the computing device in any useable form without limitation. By way of non-limiting example, if the predetermined vitreal half-life-RH relation is provided in the form of a linear regression as illustrated in FIG. 1, the predetermined vitreal half-life-RH relation may be stored in the memory of the computing device as a slope and an intercept specifying the linear regression equation. By way of another non-limiting example, if the predetermined vitreal half-life-RH relation is provided in the form of a polynomial curve fit, the predetermined vitreal half-life-RH relation may be stored in the memory as a data set that includes the degree of the polynomial of the curve fit as well as the coefficients corresponding to each term of the polynomial curve fit.

In another particular embodiment, the predetermined vitreal half-life-RH relation may be provided in the form of a linear regression as illustrated in FIG. 1. In yet another particular embodiment, the predetermined vitreal half-life-RH relation may be provided in the form of a series of at least two linear regressions, in which each linear regression is valid within a predetermined range of RH values. By way of non-limiting example, the predetermined vitreal half-life-RH relation may be defined for RH ranging between about 1 nm and about 25 nm. In this non-limiting example, the predetermined vitreal half-life-RH relation may be provided as a first linear regression to be used over RH ranging from about 1 nm to about 10 nm, and a second linear regression to be used over RH ranging from about 10 nm to about 25 nm. In yet another aspect, the predetermined vitreal half-life-RH relation may be provided in the form of a series of at least two equations specifying the predetermined vitreal half-life-RH relation over at least part of the expected range of RH regressions, in which each equation may be any of the equations including, but not limited to, linear regressions, polynomial curve fits, or any of the other equations described herein above.

In various embodiments, the predetermined vitreal half-life-RH relation may be provided in the form of one or more correlation equations in which each correlation equation is defined to be valid for values of RH ranging from about 1 nm to about 200 nm, from about 1 nm to about 100 nm, and from about 1 nm to about 50 nm. In various other embodiments, each correlation equation is defined to be valid over values of RH ranging from about 1 nm to about 45 nm, from about 1 nm to about 40 nm, from about 1 nm to about 35 nm, from about 1 nm to about 30 nm, from about 1 nm to about 25 nm, from about 1 nm to about 20 nm, from about 1 nm to about 15 nm, from about 1 nm to about 10 nm, and from about 1 nm to about 5 nm. Alternatively, the RH values may range from about 2 to about 8, from about 2 to about 6, and from about 2.5 to about 5.5. In various other embodiments, each correlation equation is defined to be valid over a subset of values of RH ranging from about 1 nm to about 3 nm, from about 2 nm to about 4 nm, from about 3 nm to about 5 nm, from about 4 nm to about 6 nm, from about 5 nm to about 7 nm, from about 6 nm to about 8 nm, from about 7 nm to about 9 nm, from about 8 nm to about 10 nm, from about 9 nm to about 11 nm, from about 10 nm to about 12 nm, from about 11 nm to about 13 nm, from about 12 nm to about 14 nm, from about 13 nm to about 15 nm, from about 14 nm to about 16 nm, from about 15 nm to about 17 nm, from about 16 nm to about 18 nm, from about 17 nm to about 19 nm, from about 18 nm to about 20 nm, from about 19 nm to about 21 nm, from about 20 nm to about 28 nm, from about 24 nm to about 32 nm, from about 28 nm to about 36 nm, from about 32 nm to about 40 nm, from about 36 nm to about 44 nm, from about 40 nm to about 48 nm, and from about 44 nm to about 50 nm.

In this regard, it is to be noted that any combination or ranges, or values within those ranges may be selected for purposes of the present method and systems without departing from the intended scope of the present disclose (e.g., about 2 to about 10, about 2.5 to about 5.5, about 3 to about 6, etc.).

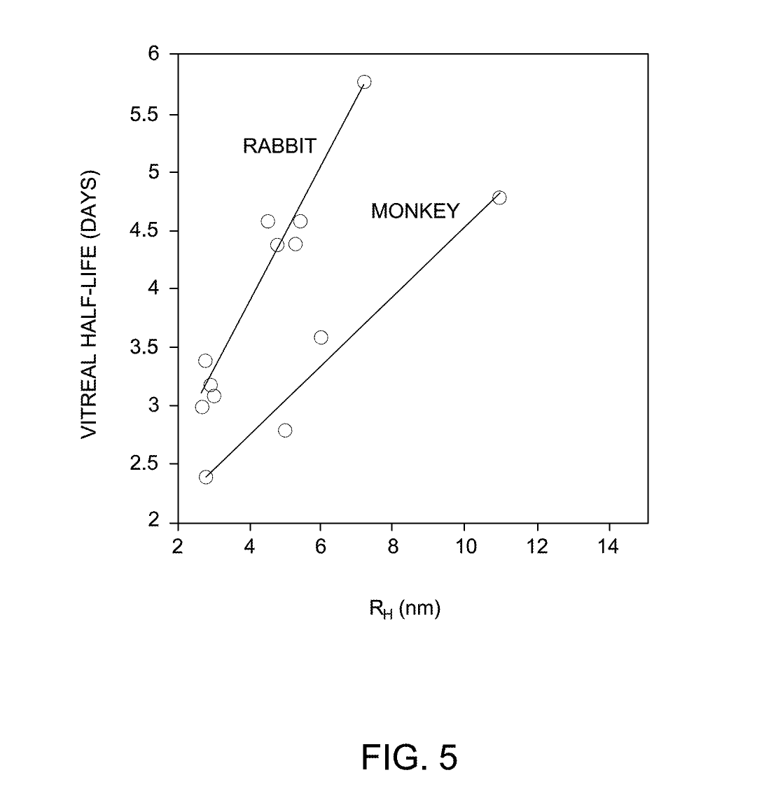

In various embodiments, the predetermined vitreal half-life-RH relation may be sensitive to one or more factors including, but not limited to: the species of the patient or animal model, the sex of the subject, the age of the subject, the morphology of the subject's eye (such as eyeball radius), and any other relevant factor. By way of non-limiting example, the composition of the vitreous humor is thought to vary between different species of animals, as well as the eye morphology, both of which may impact the predetermined vitreal half-life-RH relation. As illustrated in FIG. 5, the predetermined vitreal half-life-RH relation for a rabbit eye differs from the predetermined vitreal half-life-RH relation for a monkey eye.

In various embodiments, pharmacokinetic (PK) data used to determine vitreal half-life are collected using well-known methods and animal models. Non-limiting examples of suitable known animal models include rabbit eyes and monkey eyes. Methods of collecting and analyzing data from rabbit eyes are described herein below in Example 1. The collection and analysis of PK data from monkey eyes are similarly well known in the art, as described for example in various published articles (see, e.g., Gaudreault et al. (2005) IOVS 46:726, and Le et al. (2015) J Pharmacol Exp Ther jpet.115.227223; published ahead of print Sep. 10, 2015).

In various embodiments, a plurality of predetermined vitreal half-life-RH relations may be stored in the memory of the computing device. In these various aspects, the plurality of predetermined vitreal half-life-RH relations may be stored in association with one or more indices corresponding to one or more factors including, but not limited to, an applicable RH range, patient or animal model species, sex of patient, age of patient, a morphological parameter (such as eyeball radius), or any other factor relevant to vitreal half-life as discussed herein above. In these various embodiments, one or more additional values specifying the values of the one or more indices may be received by the computing device at step 102 of FIG. 4.

In one embodiment, for a rabbit model, the predetermined vitreal half-life-RH relation is a linear regression expressed as Eqn. (0):

Y=(1.5)+(0.6)X; Eqn. (0)

in which Y is the predicted vitreal half-life in days, X is the RH in nm, and the R² for the linear regression is greater than or equal to about 0.9.

In one embodiment, for a rabbit model, the predetermined vitreal half-life-RH relation is a linear regression expressed as Eqn. (1):

Y=(1.53±0.005)+(0.588±0.005)X; Eqn. (1)

in which Y is the predicted vitreal half-life in days, X is the RH in nm, the slope and intercept of the linear regression equation are provided as an average value±standard deviation, and the R² for the linear regression is greater than or equal to about 0.90, such as greater than or equal to about 0.95.

In another embodiment, and with reference to FIG. 3, for a rabbit model, the predetermined vitreal half-life-RH relation is a linear regression expressed as Eqn. (2):

Y=1.5322+0.58834X; Eqn. (2)

in which Y is the predicted vitreal half-life in days, X is the RH in nm, and R² of the linear regression is about 0.97434.

In another embodiment, for a monkey model, the predetermined vitreal half-life-RH relation is a linear regression expressed as Eqn. (3):

Y=(1.54±0.006)+(0.299±0.005)X; Eqn. (3)

in which Y is the predicted vitreal half-life in days, X is the RH in nm, and R² of the linear regression is greater than or equal to about 0.90, such as greater than or equal to about 0.95.

In another embodiment, for a monkey model, the predetermined vitreal half-life-RH relation is a linear regression expressed as Eqn. (4):

Y=1.5441+0.29933X; Eqn. (4)

in which Y is the predicted vitreal half-life in days, X is the RH in nm, and R² of the linear regression is about 0.98089.

In various embodiments, the predetermined vitreal half-life-RH relation is a linear regression expressed as a linear equation as described herein above with an R² value of greater than or equal to about 0.8, greater than or equal to about 0.82, greater than or equal to about 0.84, greater than or equal to about 0.85, greater than or equal to about 0.86, greater than or equal to about 0.88, greater than or equal to about 0.90, greater than or equal to about 0.91, greater than or equal to about 0.92, greater than or equal to about 0.93, greater than or equal to about 0.94, greater than or equal to about 0.95, greater than or equal to about 0.96, greater than or equal to about 0.97, greater than or equal to about 0.98, and greater than or equal to about 0.99. In another embodiment, the predetermined vitreal half-life-RH relation is a linear regression expressed as a linear equation as described herein above with an R² value of greater than or equal to about 0.9.

In other embodiments (not illustrated), the method may make use of a predetermined vitreal half-life-VH relation in a manner similar to the use of the predetermined vitreal half-life-RH relation described herein above. In these other embodiments, a hydrodynamic volume (VH) may be received and transformed into a vitreal half-life using the predetermined vitreal half-life-VH relation. The predetermined vitreal half-life-VH relation may be obtained empirically by correlating a plurality of vitreal half-lives measured for a plurality of therapeutic agent-polymer conjugates with the corresponding hydrodynamic volumes (VH) measured for the plurality of therapeutic agent-polymer conjugates in a similar manner as the correlation of vitreal half-life and hydrodynamic radius (RH) discussed herein previously.

In various embodiments, the VH of the therapeutic agent-polymer conjugate may be obtained by any known method including, but not limited to, empirically measuring the VH from a sample of the therapeutic agent-polymer conjugate; estimating VH from a chemical structure of the therapeutic agent-polymer conjugate using any known method (including, for example, estimating VH for the therapeutic agent-polymer conjugate using a known VH value for a conjugate having a substantially similar chemical structure); and retrieving a previously published VH value for the therapeutic agent-polymer conjugate. In another embodiment, the VH may be estimated by assuming that the hydrated therapeutic agent-polymer conjugate is approximately spherical in shape and calculating VH according to Eqn. (5):

VH=(4/3)π(RH)3 Eqn. (5).

In various embodiments, the predetermined vitreal half-life-VH relation may be provided in the form of one or more correlation equations in which each correlation equation is defined to be valid for values of VH ranging from about 1 nm³ to about 3.5×10⁷ nm³, from about 1 nm³ to about 4×10⁶ nm³, and from about 1 nm to about 5×10⁵ nm³. In various other embodiments, each correlation equation is defined to be valid over values of VH ranging from about 1 nm³ to about 3.8×10⁵ nm³, from about 1 nm³ to about 2.7×10⁵ nm³, from about 1 nm³ to about 1.8×10⁵ nm³, from about 1 nm³ to about 1.1×10⁵ nm³, from about 1 nm³ to about 6.5×10⁴ nm³, from about 1 nm³ to about 3.4×10⁴ nm³, from about 1 nm³ to about 1.4×10⁴ nm³, from about 1 nm³ to about 4.2×10³ nm³, and from about 1 nm³ to about 5.2×10² nm³. Alternatively, the VH values may range from about 35 nm³ to about 2150 nm³, from about 35 nm³ to about 900 nm³, and from about 65 nm³ to about 700 nm³.

Assess Predicted Vitreal Half-Life

Referring again to FIG. 4, once the predicted vitreal half-life is obtained at step 104, the method 100 may further include assessing the predicted vitreal half-life so obtained at step 106. In various embodiments, the predicted vitreal half-life is compared to the preselected vitreal half-life at step 106. In these various embodiments, if the predicted vitreal half-life is determined to be greater than or equal to the preselected vitreal half-life at step 106, the therapeutic agent-polymer conjugate may be selected for use in an ocular treatment. In various other embodiments, if predicted vitreal half-life is determined to be less than the preselected vitreal half-life at step 106, the user may elect to reject the therapeutic agent-polymer conjugate for use in an ocular treatment, or alternatively the user may elect to modify the therapeutic agent-polymer conjugate and repeat the steps 102, 104, and 106 of the method 100 using the RH of the modified therapeutic agent-polymer conjugate. In one embodiment, the user may modify the therapeutic agent-polymer conjugate by modifying the polymer moiety or by substituting a different polymer moiety. By way of non-limiting example, the user may modify a PEG polymer moiety by substituting a PEG polymer with a higher MW and RH, or a branched PEG polymer with a higher RH as the modified polymer moiety. By way of another non-limiting example, the user may substitute a different polymer, such as hyaluronic acid (HA), for the PEG polymer moiety.

In various embodiments, the preselected vitreal half-life may be selected by the user and stored in the memory of the computing device for use in step 106. The value of the therapeutic agent-polymer conjugate may be selected based on any one or more factors including, but not limited to: the ocular disorder to be treated and the amount of treatment time associated with the disorder; the composition and/or formulation of the therapeutic agent and associated pharmacokinetic, pharmacodynamic, and viscosity properties; the composition and/or formulation of the therapeutic agent-polymer conjugate, including the polymer type (PEG, HA, etc.), polymer branching, and number of arms; the desired dose and frequency of dosing of therapeutic agent to perform the ocular therapy; and any other relevant factor.

In one embodiment, the preselected vitreal half-life for the therapeutic agent-polymer conjugate may be at least twice the vitreal half-life of the therapeutic agent alone with no conjugation to a polymer moiety. In various other embodiments, the preselected vitreal half-life for the therapeutic agent-polymer conjugate may be at least 1.2-fold, at least 1.4-fold, at least 1.5-fold, at least 1.75-fold, at least 2-fold, at least 3-fold, at least 4-fold, at least 5-fold, at least 6-fold, at least 7-fold, at least 8-fold, at least 9-fold, at least 10-fold or more (e.g., at least about 20-fold, 30-fold, 40-fold, 50-fold or more) the vitreal half-life of the unconjugated therapeutic agent alone. In various other aspects, the preselected vitreal half-life for the therapeutic agent-polymer conjugate may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, and at least 15 days or more. In this regard, it is to be noted that the therapeutic agent-polymer conjugate is formulated to extend vitreal half-life, and other considerations also need to be given to challenges related to the ability to practically administer the formulation by intravitreal injection.

In one embodiment, the viscosity of the therapeutic agent-polymer conjugate may be selected in order to enable delivery of the therapeutic agent-polymer conjugate by intravitreal injection. Without being limited to any particular theory, a suitable viscosity may depend on the selected configuration of the syringe and needle and the desired injection force. In one embodiment, if a conventional syringe and a 30 gauge needle are used to inject the therapeutic agent-polymer conjugate, a suitable viscosity is less than or equal to about 200 cP, and in some embodiments may range from about 2 cP to about 200 cP, about 5 cP to about 175 cP, or about 10 cP to about 150 cP. In various other embodiments, the viscosity is less than about 1000 cP, less than about 800 cP, less than about 600 cP, less than about 500 cP, less than about 400 cP, less than about 300 cP, less than about 200 cP, less than about 100 cP, less than about 75 cP, or less than about 50 cP.

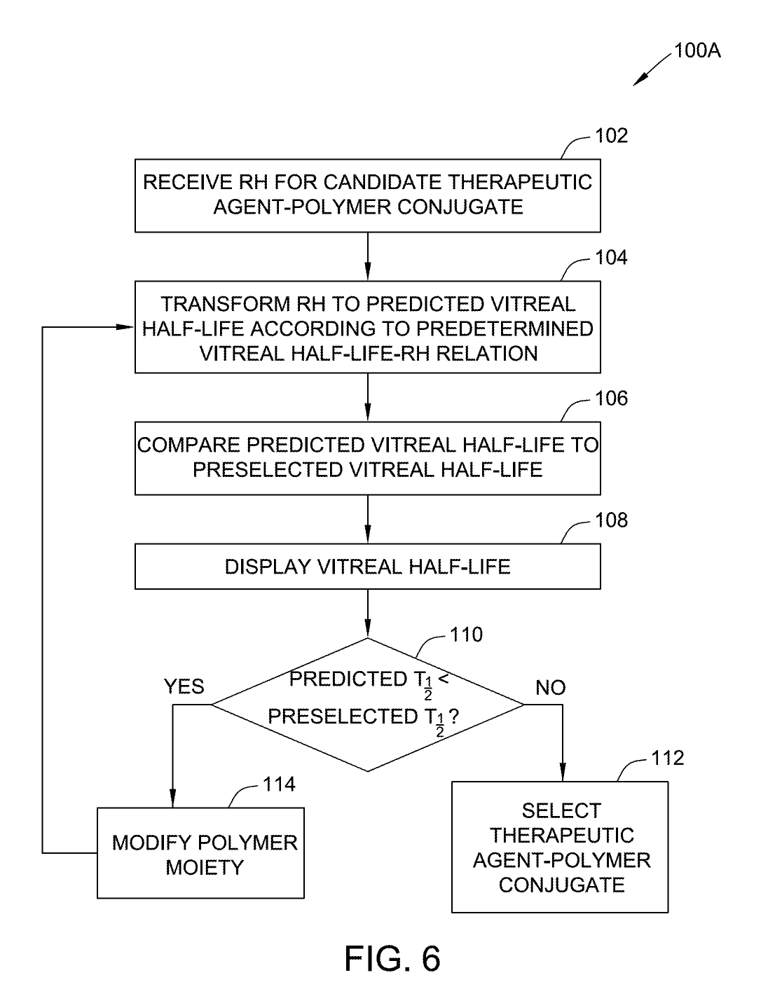

FIG. 6 is a flow chart illustrating the steps of a method 100A for identifying a therapeutic agent-polymer conjugate having a preselected vitreal half-life in another embodiment. As illustrated in FIG. 6, the comparison of the predicted vitreal half-life to the preselected vitreal half-life at step 106 may be associated with additional method steps. As illustrated in FIG. 6, comparison of the predicted vitreal half-life to the preselected vitreal half-life at step 106 may be subjected to a logical analysis at step 110. If the logical analysis at step 100 determines that the predicted vitreal half-life is greater than or equal to the preselected vitreal half-life, the therapeutic agent-polymer conjugate may be selected for use in an ocular treatment at step 112. If the logical analysis at step 100 determines that the predicted vitreal half-life is less than the preselected vitreal half-life, the therapeutic agent-polymer conjugate may be modified at step 114 to enhance the RH of the therapeutic agent-polymer conjugate and steps 102, 104, 106, and 108 of method 100A may be repeated. As further detailed elsewhere herein, such modification may be achieved by selecting a different therapeutic agent, or a different polymer, or by chemically modifying the therapeutic agent or polymer.

In various embodiments, the computer device and associated memory may include additional data, instructions, and/or features to aid in the modification of the therapeutic agent-polymer conjugate. By way of non-limiting example, the memory of the computing device may include stored data that includes predetermined estimates of RH increases due to increasing the molecular weight of a polymer moiety, due to changing the structure of the polymer moiety to a more branched structure with a comparable molecular weight, due to changing the composition of the polymer moiety from one polymer compound to another polymer compound, and any combination thereof. In this non-limiting example, the computing device may provide for the added capability of prompting a user with a list of suggested modifications to the therapeutic agent-polymer conjugate that result in a modified therapeutic agent-polymer conjugate likely to match or exceed the preselected vitreal half-life at step 114.

In various other embodiments (not illustrated) a method may include assembling a collection of candidate therapeutic agent-polymer conjugates and identifying those candidates with predicted vitreal half-lives that are greater than or equal to the preselected vitreal half-life for potential use in an ocular treatment using method 100 illustrated in FIG. 4 and/or method 100A illustrated in FIG. 6. Additionally, or alternatively, the collection of candidate therapeutic agent-polymer conjugates may be subject to testing in an animal model to determine or measure the actual in vivo vitreal half-life. Notably, and as further illustrated by Example 5 below, experience to date indicates the accuracy of the predicted vitreal half-life, based on the methods and systems of the present disclosure, as compared to the in vivo measured vitreal half-life, may in some instances be about 75%, 80%, 85%, 90%, 95% or more.

Display Results

Referring again to FIG. 4 and FIG. 6, a user interface of the computing device may display the predicted vitreal half-life at step 108. In other embodiments, step 108 may further include displaying additional information to the user including, but not limited to: identifying and/or structural information associated with the therapeutic agent-polymer conjugate, suggested modifications to the therapeutic agent-polymer conjugate as described herein above, the predetermined vitreal half-life-RH relation in either an equation form or in the form of a graph similar to the graph illustrated in FIG. 1, the molecular weight of the therapeutic agent-polymer conjugate, and any other information relevant to the results of the disclosed method.

### C. Therapeutic Agent-Polymer Conjugates

In various embodiments, the systems and methods described herein identify and/or design therapeutic agent-polymer conjugates that have greater than or equal to a predetermined vitreal half-life. As discussed herein above, the systems and methods make use of a predetermined vitreal half-life-RH relation that provides a means for transforming a hydrodynamic radius (RH) to a vitreal half-life using a correlation derived from a plurality of measured vitreal half-lives and associated hydrodynamic radii. As further discussed above, this predetermined half-life-RH relation is primarily, if not exclusively, dictated by hydrodynamic radius; stated another way, in comparison to hydrodynamic radius, this predetermined half-life-RH relation shows much less, if any, sensitivity to variations in molecular weight or charge (see, e.g., Example 4 for additional details and discussion of the net effect of charge on vitreal half-life).

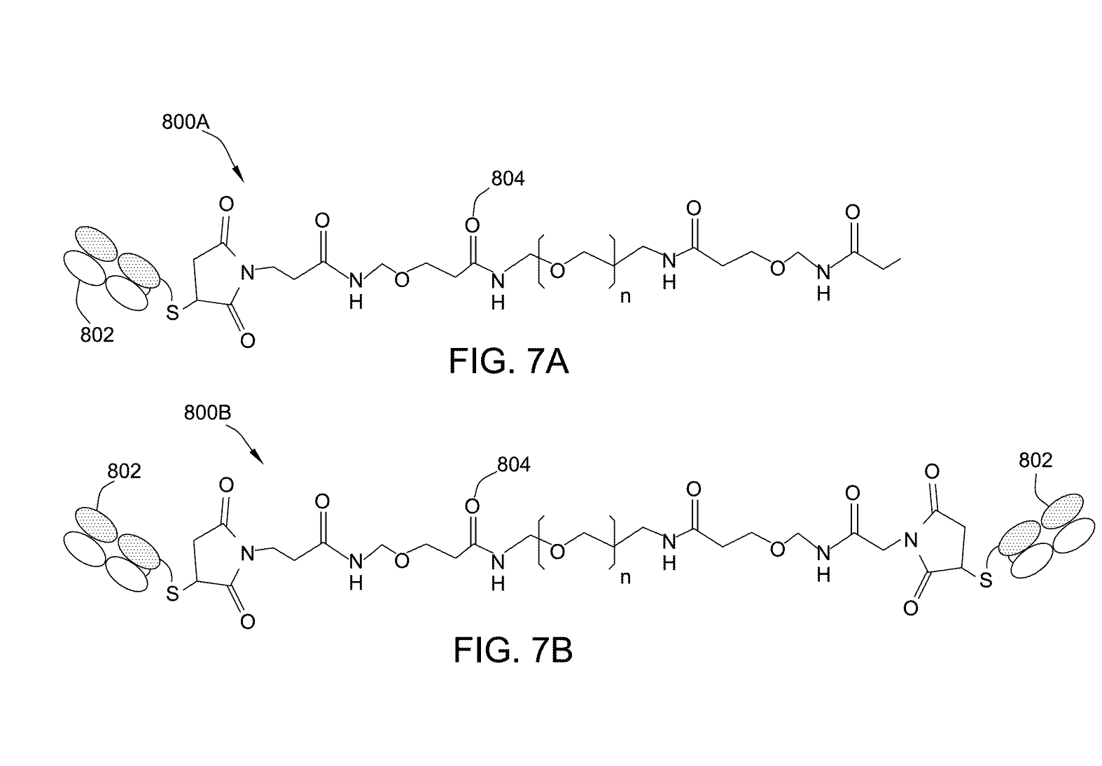

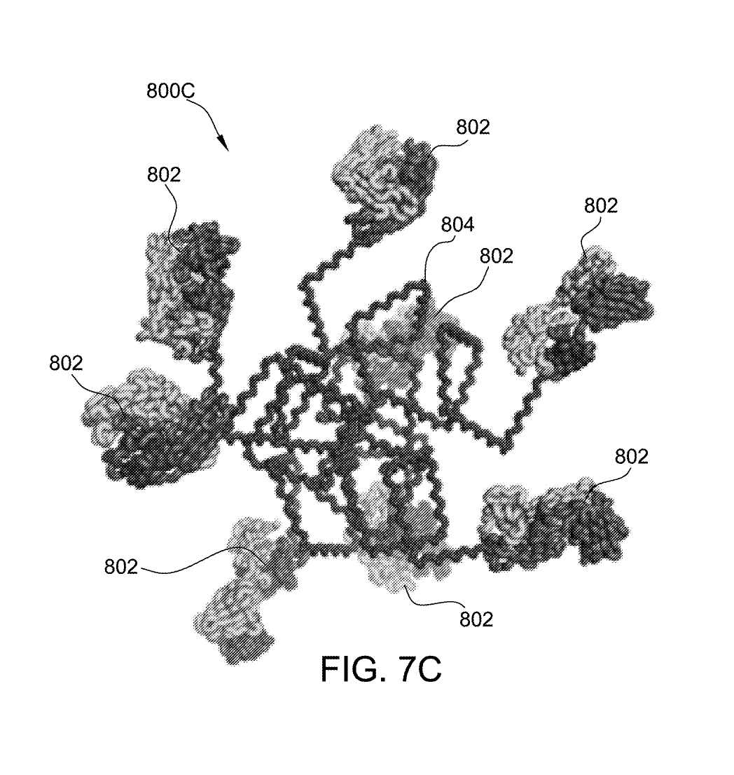

In various embodiments, the therapeutic agent-polymer conjugates include at least one therapeutic agent covalently bonded to at least part of a polymer moiety. FIGS. 7A, 7B, and 7C are illustrations of several non-limiting examples of therapeutic agent-polymer conjugates 800A, 800B, and 800C. Referring to FIG. 7A, the therapeutic agent-polymer conjugate 800A may include a single therapeutic agent 802 covalently bonded to a polymer moiety 804. Referring to FIG. 7B, the therapeutic agent-polymer conjugate 800B may include at least two therapeutic agents 802/802A covalently bonded to a polymer moiety 804. In various aspects, the polymer moiety 804 may be a linear polymer, as illustrated in FIGS. 7A and 7B, or the polymer moiety may be branched, as illustrated in FIG. 7C. Referring to FIG. 7C, the therapeutic agent-polymer conjugate 800C may include at least two therapeutic agents 802/802A covalently bonded to a branched or multi-arm polymer moiety 804.

The polymer may be covalently bonded to the therapeutic agent 802 to form the polymer moiety 804 using any method known in the art. By way of one non-limiting example, a PEG polymer may be covalently bonded to rabFab, a non-immunogenic surrogate compound for the evaluation of ocular PK in a rabbit model, using the method described in Example 1 below. In another non-limiting example, hyaluronic acid may be covalently bonded to an anti-VEGF antibody using the methods described in U.S. Patent Application Publication No. 2011/006417, which is hereby incorporated by reference in its entirety. In yet another non-limiting example, a protein or protein fragment that binds hyaluronic acid in vivo in the vitreous may be linked to an anti-VEGF antibody using the methods described in U.S. Patent Application Publication No. 2014/0186350, which is hereby incorporated by reference in its entirety.

In various embodiments, the therapeutic agent-polymer conjugates have a hydrodynamic radius (RH) ranging from about 1 nm to about 200 nm, from about 1 nm to about 100 nm, or from about 1 nm to about 50 nm. In various other embodiments, therapeutic agent-polymer conjugates have a hydrodynamic radius (RH) ranging from about 1 nm to about 45 nm, from about 1 nm to about 40 nm, from about 1 nm to about 35 nm, from about 1 nm to about 30 nm, from about 1 nm to about 25 nm, from about 1 nm to about 20 nm, from about 1 nm to about 15 nm, from about 1 nm to about 10 nm, or from about 1 nm to about 5 nm.

Therapeutic Agents

In various embodiments, the therapeutic agent may be any compound suitable for use in ocular treatments without limitation. Non-limiting examples of suitable therapeutic agents include proteins, protein fragments, fusion proteins, antibodies, antibody fragments, and small molecules. Non-limiting examples of antibodies include monoclonal antibodies that inhibit tumor necrosis factor (TNF), epithelial growth factor receptor, vascular endothelial growth factor (VEGF), basic fibroblast growth factor receptor, CD11a, B-lymphocyte antigen CD20, CD25, CD52, and platelet-derived growth factor receptor. Non-limiting examples of anti-VEGF antibodies include ranibizumab and bevacizumab. A non-limiting example of an exemplary fusion protein that inhibits VEGF is aflibercept. Non-limiting examples of anti-TNF antibodies include infliximab, etanercept and adalimumab. Non-limiting examples of anti-CD11a antibodies include efalizumab. Non-limiting examples of anti-CD20 antibodies include rituximab. Non-limiting examples of anti-CD25 antibodies include daclizumab. Non-limiting examples of anti-CD52 antibodies include alemtuzumab. Exemplary small molecules include steroidal anti-inflammatory compounds such as triamcinolone, PI3K inhibitors such as LY294002 and m-TOR inhibitors such as Palomid 529.

Polymer Moieties

In various embodiments, suitable polymer moieties for inclusion in a therapeutic agent-polymer conjugates may include any water-soluble high molecular weight compound that has a hydrodynamic radius sufficient to acceptably increase the vitreal half-life of a therapeutic agent-polymer conjugate. In one embodiment, any known measurement method including, but not limited to, dynamic light scattering can be used to measure the hydrodynamic radius of the polymer moieties, and the therapeutic agent-polymer conjugates containing the polymer moieties.

Non-limiting examples of particularly useful polymer moieties include: polysaccharides, such as glycosaminoglycans, hyaluronans, and alginates, polyesters, high molecular weight polyoxyalkylene ether (such as PLURONIC™), polyamides, polyurethanes, polysiloxanes, polyacrylates, polyols, polyvinylpyrrolidones, polyvinyl alcohols, polyanhydrides, carboxymethyl celluloses, other cellulose derivatives, Chitosan, polyaldehydes or polyethers.

In one embodiment, the polymer moieties are sufficiently soluble in water or physiological solutions. In addition, the polymer moieties may have molecular weights ranging up to about 500,000 D, and preferably is at least about 20,000 D, or at least about 30,000 D, or at least about 40,000 D. The molecular weight chosen can depend upon the effective size of the conjugate to be achieved, the nature (e.g., structure, such as linear or branched) of the polymer, and the degree of derivatization, i.e. the number of polymer moieties per antibody fragment, and the polymer attachment site or sites on the antibody fragment. The polymer moieties may have a hydrodynamic radius of sufficient size to suitably enhance the vitreal half-life of the therapeutic agent-polymer conjugate relative to the therapeutic agent in isolation.

In one embodiment the polymer moieties have a hydrodynamic radius ranging from about 0.5 nanometers (nm) to about 100 nm, or from about 2 nm to about 8 nm. In another embodiment the polymer moieties have a hydrodynamic radius ranging from about 1 nm to about 500 nm. In another embodiment the polymer moieties have a hydrodynamic radius ranging from about 1 nm to about 200 nm. In another embodiment the polymer moieties have a hydrodynamic radius ranging from about 1 nm to about 100 nm. In another embodiment the polymer moieties have a hydrodynamic radius ranging from about 1 nm to about 50 nm. In another embodiment the polymer moieties have a hydrodynamic radius ranging from about 1 nm to about 10 nm. In another embodiment, the polymer moieties have a hydrodynamic radius of about 4 nm. In an additional embodiment, the polymer moieties have a hydrodynamic radius of about 8 nm. In another additional embodiment, the polymer moieties have a hydrodynamic radius of about 12 nm.

In one embodiment, the polymer moiety is a polyether polyol. In one embodiment, the polymer moiety is a polyethylene glycol (PEG), a polypropylene glycol (PPG), or a copolymer comprising polyethylene glycol and polypropylene repeat units. In one embodiment, the polymer comprises polyethylene glycol (PEG). PEG may have a free hydroxyl group or may be alkylated. In another embodiment, the terminal end of the PEG not bound to the therapeutic agent has a methoxy group (mPEG). Polyethylene glycols may be linear, branched, or multi-armed. In some embodiments, the PEG may be multi-armed. In some embodiments, the PEG comprises a multi-arm PEG selected from a 2-armed PEG, a 3-armed PEG, a 4-armed PEG, a 5-armed PEG, a 6-armed PEG, a 7-armed PEG, an 8-armed PEG, a 9-armed PEG, a 10-armed PEG, an 11-armed PEG, and a 12-armed PEG. In some embodiments, the multi-arm PEG is selected from a 4-armed PEG, a 6-armed PEG, and an 8-armed PEG. In some embodiments, the polyethylene glycols may comprise from about 3 repeat units to about 4000 repeat units, such as from about 20 repeat units to about 2000 repeat units, such as from about 100 repeat units to about 1000 repeat units, such as about 450 repeat units. In one embodiment, PEG has a molecular weight of from about 800 Daltons (Da) to about 100,000 Da. In further embodiments, the polyethylene glycol is a 20 kDa PEG, 40 kDa PEG, or 80 kDa PEG.

In another embodiment the polymer moiety is a polysaccharide. In one embodiment, the soluble, high molecular weight steric group is dextran. Dextran may be linear or branched. In one embodiment, the dextran is a carboxymethyl dextran (CMDex).

In another embodiment the polymer moiety is a cellulose derivative. In another embodiment the polymer moiety is a carboxymethyl cellulose (CMC). CMC, an analog of dextran, and its reducing end is available for coupling to an amine group of a biologically active compound by the Schiff-Base chemistry in conjugation. In another embodiment the polymer moiety is a polyglucosamine. In another embodiment the polymer moiety is a chitosan.

Polysaccharides may be attached to an amine at a terminus of the therapeutic agent by reductive amination. Polysaccharides containing a reducing terminus such as an aldehyde or hemiacetal functionality may be conjugated to a primary amine-containing therapeutic agent by reductive amination to afford a secondary amine linkage. Alternately, a therapeutic agent may be modified such that a covalent linkage exists between the therapeutic agent and a hydrazine or hydrazide functionality. The formation of an imine with either of these amine equivalents provides a conjugate that is stabilized to hydrolysis relative to a conventional imine. The hydrazine or hydrazide couplings are useful when the reductive amination is limited by the length of the linker. For example, a hydrazine or hydrazide coupling is especially useful when a linker is needed to separate a bulky polymer moiety and a high electron density macromolecule therapeutic agent, while allowing the reactive group of each moiety to come together. The linker between an oligonucleotide amine and the hydrazine or hydrazide may afford an extra measure of steric freedom. The imine that results from a hydrazine or hydrazide may be used without further reduction or reduced to afford an amine-like linkage.

In another embodiment, the polymer moiety is a polyaldehyde. In further embodiments, the polyaldehyde group may be either synthetically derived or obtained by oxidation of an oligosaccharide.

In another embodiment the polymer moiety is an alginate. In a preferred embodiment, the alginate group is an anionic alginate group that is provided as a salt with a cationic counter-ion, such as sodium or calcium.

In another embodiment the polymer moiety is a polyester. In particular embodiments the polyester group may be a co-block polymeric polyesteric group.

In another embodiment the polymer moiety is a polylactic acid (PLA) or a polylactide-co-glycolide (PLGA). Suitable PLGA groups and method s for conjugating PLGA groups are found in J. H. Jeong et al., Bioconjugate Chemistry 2001, 12, 917-923; J. E. Oh et al., Journal of Controlled Release 1999, 57, 269-280 and J. E. Oh et al., U.S. Pat. No. 6,589,548; the contents of each are hereby incorporated by reference in their entirety.

In another embodiment, the polymer moiety is a dendron. The dendron may be composed of any combination of monomer and surface modifications. Examples of useful monomers include, but are not limited to, polyamidoamine (PAMAM). Examples of useful surface modification groups include, but are not limited to, cationic ammonium, N-acyl, and N-carboxymethyl group. The dendron may be polyanionic, polycationic, hydrophobic or hydrophilic. In one particular embodiment, the dendron has about 1 to about 256 surface modification groups. In another particular embodiment, the dendron has about 4, 8, 16, 32, 64 or 128 surface modification groups. Examples of dendron and dendrimer conjugation techniques are found in U.S. Pat. No. 5,714,166; which is hereby incorporated by reference in its entirety.

In another embodiment, the polymer moiety is bovine serum albumin (BSA). The presence of free thiol on BSA permits the conjugation of amine-containing therapeutic agents to BSA by employing a bifunctional linker that contains a thiol-reactive group on one terminus and an amine-reactive group on the other terminus.

In other embodiments the polymer moiety may be a glycosaminoglycan, a hyaluronan, a hyaluronic acid (HA), an alginate a high molecular weight polyoxyalkylene ether (such as Pluronic™), a polyamide, a polyurethane, a polysiloxane, a polyacrylate, a polyvinylpyrrolidone, a polyvinyl alcohol, a polyanhydride, a polyether or a polycaprolactone. In other embodiments, the polymer moiety may be a hydroxyethyl starch (HES) or a 2-polyalkyloxazoline (POZ). In other embodiments, the polymer moiety may be a heparosan. In other embodiments, the polymer moiety may be a phosphorylcholine polymer.

### D. Systems and Devices for Identifying Therapeutic Agent-Polymer Conjugate with Preselected Vitreal Half-Life

Described herein are computer systems such as computing devices and user computer systems. As described herein, all such computer systems include a processor and a memory. However, any processor in a computer device referred to herein may also refer to one or more processors wherein each processor may be in one computing device or a plurality of computing devices acting in parallel. Additionally, any memory in a computer device referred to herein may also refer to one or more memories wherein the memories may be in one computing device or a plurality of computing devices acting in parallel.

As used herein, a processor may include any programmable system including systems using micro-controllers, reduced instruction set circuits (RISC), application specific integrated circuits (ASICs), logic circuits, and any other circuit or processor capable of executing the functions described herein. The above are examples only, and are thus not intended to limit in any way the definition and/or meaning of the term “processor.”

As used herein, the term “database” may refer to either a body of data, a relational database management system (RDBMS), or to both. As used herein, a database may include any collection of data including hierarchical databases, relational databases, flat file databases, object-relational databases, object oriented databases, and any other structured collection of records or data that is stored in a computer system. The above examples are example only, and thus are not intended to limit in any way the definition and/or meaning of the term database. Examples of RDBMS's include, but are not limited to including, Oracle® Database, MySQL, IBM® DB2, Microsoft® SQL Server, Sybase®, and PostgreSQL. However, any database may be used that enables the systems and methods described herein. (Oracle is a registered trademark of Oracle Corporation, Redwood Shores, Calif.; IBM is a registered trademark of International Business Machines Corporation, Armonk, N.Y.; Microsoft is a registered trademark of Microsoft Corporation, Redmond, Wash.; and Sybase is a registered trademark of Sybase, Dublin, Calif.)

In one embodiment, a computer program is provided, and the program is embodied on a computer readable medium. In an example embodiment, the system is executed on a single computer system, without requiring a connection to a server computer. In a further embodiment, the system is executed in a Windows® environment (Windows is a registered trademark of Microsoft Corporation, Redmond, Wash.). In yet another embodiment, the system is executed in a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium.

The systems and processes are not limited to the specific embodiments described herein. In addition, components of each system and each process can be practiced independent and separate from other components and processes described herein. Each component and process also can be used in combination with other assembly packages and processes.

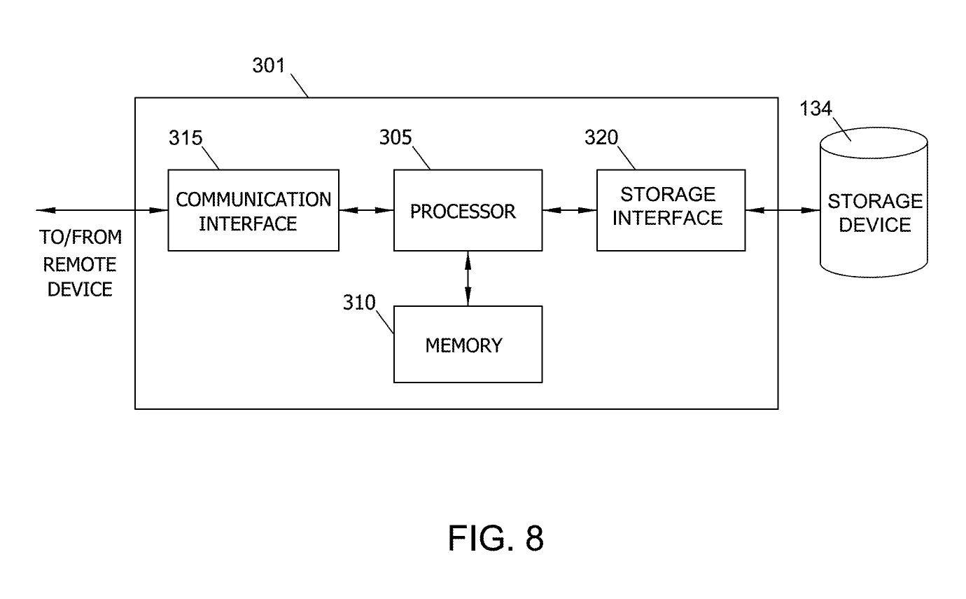

In one embodiment, the system may be configured as a server system. FIG. 8 illustrates an example configuration of a server system 301 such as a computing device used to receive the RH, transform the RH to a predicted vitreal half-life, assess the predicted vitreal half-life, and display the predicted vitreal half-life on an interactive user interface as described herein above and illustrated in FIG. 8 in one embodiment. Server system 301 may also include, but is not limited to, a database server. In the example embodiment, server system 301 performs all of the steps of the method described herein above.

Server system 301 includes a processor 305 for executing instructions. Instructions may be stored in a memory area 310, for example. Processor 305 may include one or more processing units (e.g., in a multi-core configuration) for executing instructions. The instructions may be executed within a variety of different operating systems on the server system 301, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C#, C++, Java, or other suitable programming languages, etc.).

Processor 305 is operatively coupled to a communication interface 315 such that server system 301 is capable of communicating with a remote device such as a user system or another server system 301. For example, communication interface 315 may receive requests (e.g., requests to provide an interactive user interface to receive RH inputs and to display the predicted vitreal half-life) from a client system via the Internet.

Processor 305 may also be operatively coupled to a storage device 134. Storage device 134 is any computer-operated hardware suitable for storing and/or retrieving data. In some embodiments, storage device 134 is integrated in server system 301. For example, server system 301 may include one or more hard disk drives as storage device 134. In other embodiments, storage device 134 is external to server system 301 and may be accessed by a plurality of server systems 301. For example, storage device 134 may include multiple storage units such as hard disks or solid state disks in a redundant array of inexpensive disks (RAID) configuration. Storage device 134 may include a storage area network (SAN) and/or a network attached storage (NAS) system.

In some embodiments, processor 305 is operatively coupled to storage device 134 via a storage interface 320. Storage interface 320 is any component capable of providing processor 305 with access to storage device 134. Storage interface 320 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 305 with access to storage device 134.

Memory area 310 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

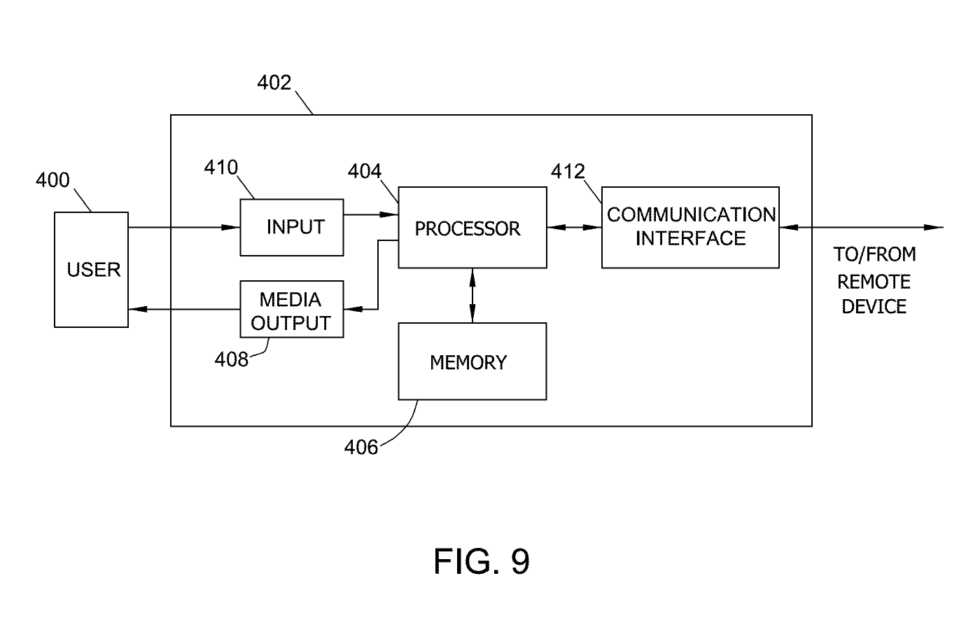

In another embodiment, the system may be provided in the form of a computing device. FIG. 9 illustrates an example configuration of a computing device 402. Client computing device 402 includes a processor 404 for executing instructions. In some embodiments, executable instructions are stored in a memory area 406. Processor 404 may include one or more processing units (e.g., in a multi-core configuration). Memory area 406 is any device allowing information such as executable instructions and/or other data to be stored and retrieved. Memory area 406 may include one or more computer-readable media.

In another embodiment, the memory area 406 included in the computing device 402 of the system for identifying a therapeutic agent-polymer conjugate having a preselected vitreal half-life may include a plurality of modules (not illustrated). Each module may include instructions configured to execute using at least one processor 404. The instructions contained in the plurality of modules may implement at least part of the method for identifying a therapeutic agent-polymer conjugate having a preselected vitreal half-life described herein above when executed by the one or more processors 404 of the computing device. Non-limiting examples of modules stored in the memory area 406 of the computing device include: a first module to receive a hydrodynamic radius (RH) of the therapeutic agent-polymer conjugate; a second module to transform the RH to a predicted vitreal half-life of the therapeutic agent-polymer conjugate according to a predetermined vitreal half-life-RH relation; a third module to assess whether the predicted vitreal half-life is at least the preselected vitreal half-life; a fourth module to display, on a user interface of the computing device, the therapeutic agent-polymer conjugate comprising the therapeutic agent and the modified polymer moiety, and the predicted vitreal half-life; a fifth module to modify the polymer moiety of the therapeutic agent-polymer conjugate to increase the RH if the predicted vitreal half-life is less than the preselected vitreal half-life, and to re-execute the instructions of the first, second, third, and fourth modules until the predicted vitreal half-life of the conjugate is greater than or equal to the preselected vitreal half-life; and any combination thereof.

Computing device 402 also includes one media output component 408 for presenting information to a user 400. Media output component 408 is any component capable of conveying information to user 400. In some embodiments, media output component 408 includes an output adapter such as a video adapter and/or an audio adapter. An output adapter is operatively coupled to processor 404 and is further configured to be operatively coupled to an output device such as a display device (e.g., a liquid crystal display (LCD), organic light emitting diode (OLED) display, cathode ray tube (CRT), or “electronic ink” display) or an audio output device (e.g., a speaker or headphones).

In some embodiments, client computing device 402 includes an input device 410 for receiving input from user 400. Input device 410 may include, for example, a keyboard, a pointing device, a mouse, a stylus, a touch sensitive panel (e.g., a touch pad or a touch screen), a camera, a gyroscope, an accelerometer, a position detector, and/or an audio input device. A single component such as a touch screen may function as both an output device of media output component 408 and input device 410.

Computing device 402 may also include a communication interface 412, which is configured to communicatively couple to a remote device such as server system 301 (see FIG. 8) or a web server. Communication interface 412 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 406 are, for example, computer-readable instructions for providing a user interface to user 400 via media output component 408 and, optionally, receiving and processing input from input device 410. A user interface may include, among other possibilities, a web browser and an application. Web browsers enable users 400 to display and interact with media and other information typically embedded on a web page or a website from a web server. An application allows users 400 to interact with a server application. The user interface, via one or both of a web browser and an application, facilitates display of information such as the predicted vitreal half-life generated by the computing device 402.

The present disclosure uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Examples

### Example 1: Vitreal Pharmacokinetics of rabFab Surrogate Compound

Development of delivery technologies for protein therapeutic agents requires testing in relevant animal models to demonstrate in vivo utility. Rabbit models are commonly employed during early studies of ocular pharmacokinetics. Unfortunately, most human and humanized antibodies are immunogenic in rabbits, precluding estimation of key pharmacokinetic parameters using long acting delivery technologies. To address this problem, rabbit Fab (“rabFab”), a species-matched surrogate compound, was developed for evaluating delivery technologies in rabbit models. The rabFab described herein was derived from a rabbit monoclonal antibody that binds to human phosphor c-Met.

Methods of making rabbit antibodies, including rabbit monoclonal antibodies, are well known in the art. See, for example, U.S. Pat. Nos. 5,675,063 and/or 7,429,487. An exemplary rabbit monoclonal antibody that binds to human phospho c-Met is commercially available from Abcam (Cambridge, Mass., USA), product number ab68141.

A study of the ocular pharmacokinetics of rabFab upon intravitreal injection (0.3 mg dose) in rabbits (New Zealand Whites) was performed.

rabFab (rabbit anti-cMet Fab) produced by bioengineered CHO cell cultures was purified from conditioned CHO media using a three column step process: affinity, cation exchange and gel filtration.

Naïve New Zealand White (NZW) rabbits (3.1 kg to 4.1 kg and approximately 4 months of age at the time of dosing) were assigned to dose groups and dosed with Anti-phospho cMet Fab. The anti-phospho cMet Fab was administered via a single bilateral intravitreal injection to the rabbits followed by up to 27 days of observation. Topical antibiotic (tobramicin ophthalmic ointment) was applied to both eyes twice on the day before treatment, immediately following the injection, and twice on the day following the injection, with the exception of animals sent to necropsy on Days 1 and 2. Prior to dosing, mydriatic drops (1% tropicamide) were applied to each eye for full pupil dilation. Animals were sedated with isoflurane/oxygen gas prior to and during the procedure. Alcaine (0.5%) was also applied to each eye prior to injection. The conjunctivae were flushed with benzalkonium chloride (Zephiran) diluted in sterile water, U.S.P. to 1:10,000 (v/v).

Syringes were filled under a laminar flow hood immediately prior to dosing. Anti-phospho cMet Fab was administered by a single 30 μL intravitreal injection (0.3 mg dose) to both eyes in all animals. Doses were administered by a board-certified veterinary ophthalmologist using sterilized 100 μL Hamilton Luer Lock syringes with a 30-gauge×½″ needle. In order to mimic clinical dosing, eyes were dosed in the infero-temporal quadrants, i.e. in 5 o'clock and 7 o'clock positions for the left and right eyes, respectively (when facing the animal). The eyes were examined by slit-lamp biomicroscopy and/or indirect ophthalmoscopy immediately following treatment.

All animals underwent exsanguination by incision of the axillary or femoral arteries following anesthesia by intravenous injection of sodium pentobarbital. Aqueous humor, vitreous humor and retina tissue were collected, snap frozen in liquid nitrogen and stored at −80° C. Determination of vitreous concentrations of test article was by antigen-binding ELISA. Values below the LLOQ were not used in pharmacokinetic analysis or for graphical or summary purposes.

An ELISA analysis was performed using a target-coat method. In this assay, the target-coat was phosphorylated cMet peptide conjugated to KLH (P-cMet peptide, from Yenzym, South San Francisco, Calif.). For preparation of assay plates, lyophilized P-cMet peptide was reconstituted with 300 μl of buffer and further diluted to 1:200 in 0.05M sodium bicarbonate buffer. The diluted P-cMet peptide (100 μL/well) was added to a 96 well microtiter plate (Nunc, Thermo Scientific, Rockford, Ill.) and incubated overnight at 2-8° C. After incubation the plate was washed three times with 400 μl of wash buffer (BA029), followed by blocking, with assay diluent (wash buffer containing 0.5% bovine serum albumin and 0.05% Proclin). A standard curve was prepared by diluting Rabbit Fab (Genentech, South San Francisco, Calif.) to 200 ng/ml and then 1:2 serial dilution in assay diluent. The controls were diluted 1:100 in assay diluent. Each sample was diluted to the quantitative range of assay using assay diluent. All samples, controls and standards were added to the plate at 100 μl and incubated at room temperature for 2 hrs. with gentle agitation. After incubation and washing, 100 μl of the detection antibody (mouse anti-rabbit light chain-HRP, SouthernBiotech, Birmingham, Ala.) was added per well, after a 1/4,000 dilution in assay diluent. Plates were then incubated for 1 hr. at room temperature with gentle agitation. After additional washing, 100 μl of HRP substrate (3,3′,5,5′-tetramethylbenzidine, TMB, from Kirkegard & Perry Laboratory, Gaithersburg, Md.) was added to each well, followed by 15 min incubation at room temperature with gentle agitation. Each reaction was stopped with 100 μl of 1M phosphoric acid. The plate was read at a wavelength of 450 nm for detection and at a wavelength of 630 nm as a reference measurement (SpectraMax 384-plus; Molecular Devices, Sunnyvale, Calif.). The optical density values of the standards were plotted using a four-parameter logistic curve-fitting software (Softmax, Molecular Devices), from which concentration values for controls and test samples were derived by extrapolation.

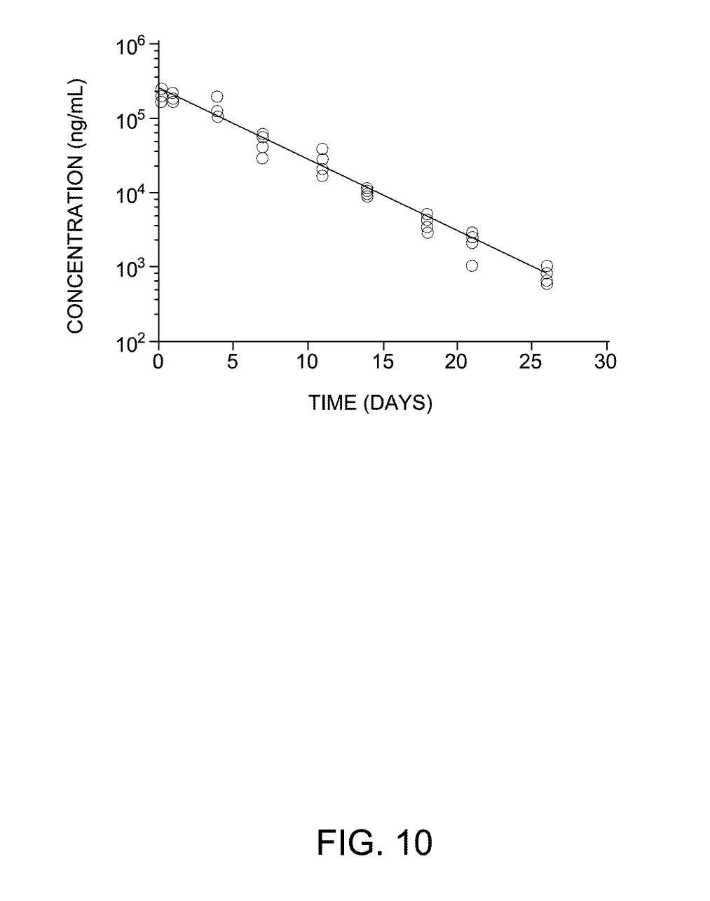

Concentrations in the vitreous humor were determined by antigen-binding ELISA as described above and plotted as a function of time. FIG. 10 is a graph summarizing the vitreal concentrations as a function of time post-injection. These data were subjected to a non-compartmental analysis to obtain pharmacokinetic (PK) parameters.

The pharmacokinetic parameters were determined by one-compartmental analysis with nominal time and dose (Phoenix WinNonlin version 6.4, Pharsight Corp, Sunnyvale, Calif.). To estimate single dose vitreous PK parameters, a one-compartmental IV bolus dosing model was used with 1/Y² weighting and with nominal time and dose. PK parameters calculated using the non-compartmental analysis are summarized in Table 1 below, with 95% confidence intervals indicated in parentheses. The vitreous concentration versus time profile was well described by first-order elimination kinetics. Although these samples were not specifically tested for anti-therapeutic antibody (ATA) response, the concentration versus time curve and small inter-animal variability suggested an absence of an immune response against rabFab in rabbits. The calculated Cmax and VSS values were consistent with the dose administered and the dimensions of the rabbit eye, respectively. The vitreal half-life and clearance (CL) were similar to corresponding values measured for other antibody Fab fragments in rabbits upon intravitreal injection.

TABLE 1
Pharmacokinetic Parameters Estimated Using One-compartmental Analysis

| | Vitreous Tissue | | | | |
|---|---|---|---|---|---|
| | Cₘₐₓ | Half-life | AUCₐₗₗ | CL | V_{SS} |
| Test Article | (μg/mL) | (day) | (day * μg/mL) | (mL/day) | (mL) |
| anti-phospho | 202 | 3.2 | 925 | 0.32 | 1.5 |
| cMetFab | (184, 220) | (2.9, 3.4) | (851, 1000) | (0.30, 0.35) | (1.4, 1.6) |
| 20K-anti-phospho | 166 | 4.7 | 1116 | 0.27 | 1.8 |
| cMetFab | (156, 176) | (4.4, 4.9) | (1059, 1173) | (0.26, 0.28) | (1.7, 1.9) |
| 40K-anti-phospho | 169 | 6.1 | 1478 | 0.20 | 1.8 |
| cMetFab | (153, 184) | (5.5, 6.7) | (1361, 1596) | (0.19, 0.22) | (1.6, 1.9) |

To assess consistency of PK upon extended exposure to rabbit Fab, a repeat dosing study was conducted. Animals were dosed and vitreal tissues were harvested as described above except that the 30 μL intravitreal injections (0.3 mg doses) of rabbit Fab solution were administered to both eyes of each animal by ITV injection at days 0, 12, and 24. Samples were collected at 3 hours, 6 days, 12 days (prior to dose), 12 days (3 hours post 2^{nd} dose), 18 days, 24 days (prior to dose), 24 days (3 hours post 3^{rd} dose), 31 days, and 38 days. Rabbit Fab concentrations were determined as described above and compared to predicted concentrations which were calculated using Phoenix WinNonlin with the PK parameter estimates obtained from the single dose study.

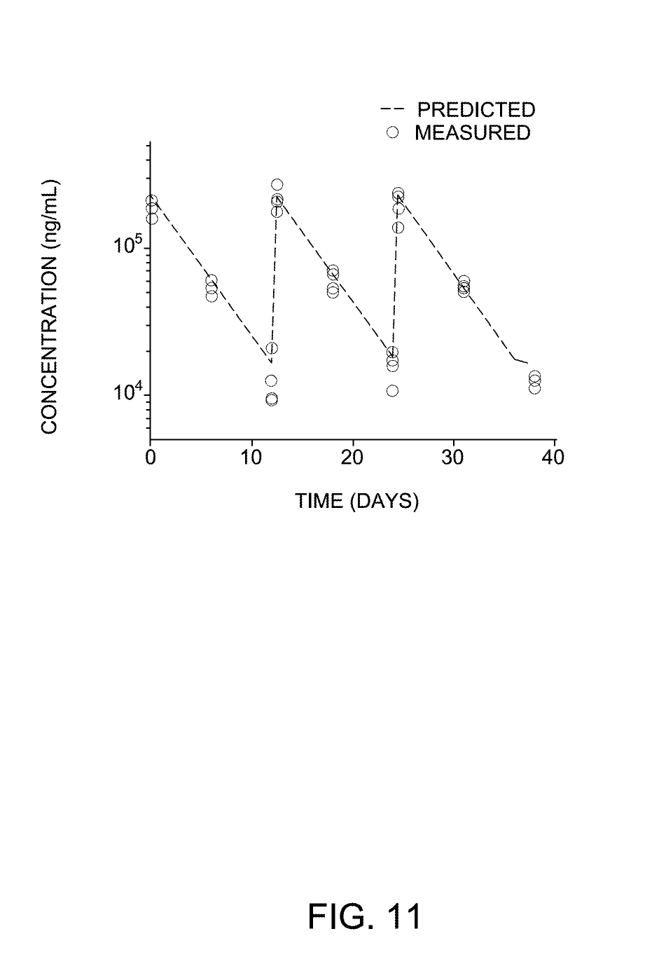

The vitreal concentration profiles measured using the ELISA assay as described above are summarized in the graph of FIG. 11. The concentration profile expected on the basis of the single dose study was consistent with the profile measured upon repeat dosing suggesting that extended and repeat exposure to a 0.3 mg dose did not result in altered pharmacokinetics. From these data it may be concluded that rabFab is an appropriate test article for evaluating ocular pharmacokinetics of Fab fragments in rabbit.

### Example 2: Vitreal Pharmacokinetics of Surrogate-Polymer Conjugate

To further evaluate suitability of rabFab as a surrogate for testing technologies to improve vitreal pharmacokinetics, PEGylated versions of the rabFab molecule were produced as described below and subjected to PK testing using the methods described in Ex. 1. In order to preserve binding activity of the rabFab molecule, site-specific coupling was performed using PEG-maleimide to modify the free Cys (Cys-227) in the Fab′ version of the rabbit antibody. Linear PEG chains of 20,000 Da and 40,000 Da molecular weight were conjugated to rabFab to produce PEGylated (20 kD) anti-phospho cMet Fab conjugate and PEGylated (40 kD) anti-phospho cMet Fab conjugate.

Rabbit Fab′ was dialyzed against PBS, pH 7.4 and then EDTA was added to a final concentration of 5 mM. To remove free thiol adducts, fresh DTT was added at a molar ratio of 1:1.2 (Fab′:DTT) and the sample was allowed to sit at room temperature overnight. Adduct removal was confirmed by LC-MS. Following reduction, polyethylene glycol maleimide (PEG-mal) from NOF America Corporation having a molecular weight of either 20 kD (Sunbright ME-200MA0B) or 40 kD (Sunbright ME-400MA) was diluted in water and added to the Fab′ pool at a molar ratio of 1:3 (Fab′:PEG). The reaction was gently rotated overnight and progress monitored by LC-MS. Removal of contaminants was performed by cation exchange using a 5 mL GE Healthcare SP HP column. The column was washed with 5 CVs of 25 mM sodium acetate pH 5.0 then eluted with 1 M NaCl over 30 CVs. Fractions (0.5 mL) were collected and peak fractions were separated by 4-20% Tris-Glycine SDS-PAGE to analyze purity and pooled accordingly.

For the samples containing 20 kD and 40 kD PEGylated rabFab′ conjugates (Genentech, South San Francisco, Calif.), the ELISA assay used was essentially the same as described above in Ex. 1, with the following modifications: i) samples and controls were diluted 1:20 prior to testing; and ii) substrate was used at a 1/10,000 dilution; and iii) standard curve started at 200 ng/mL.

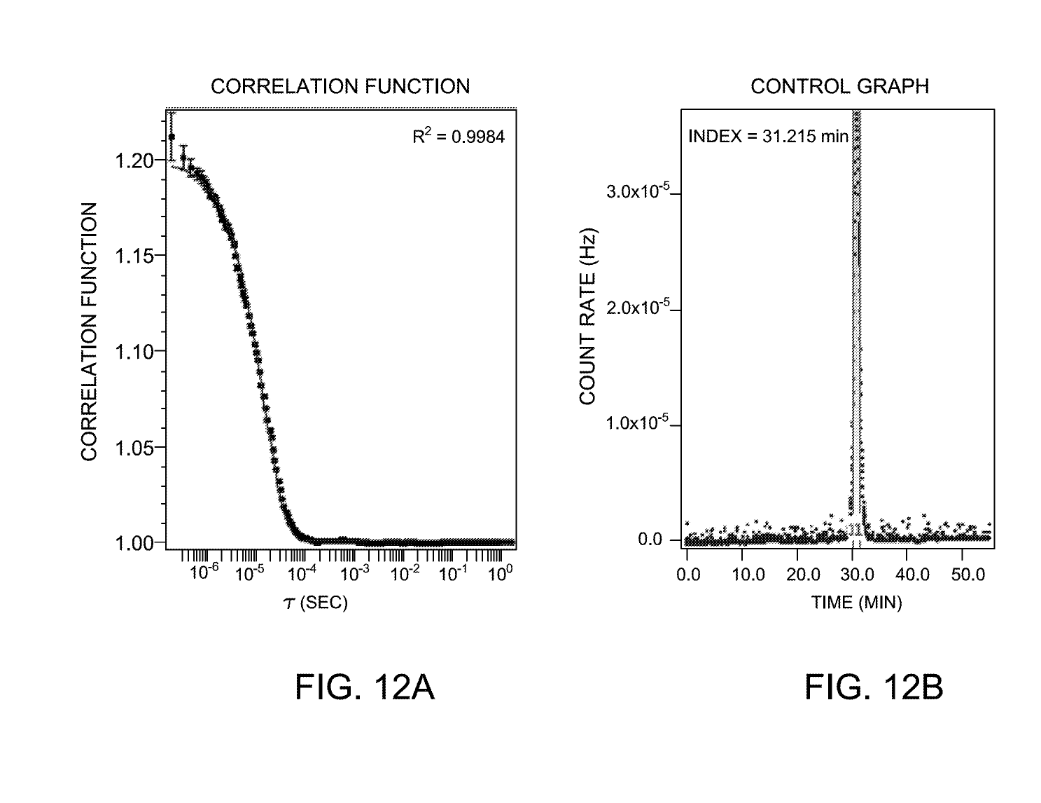

In addition, hydrodynamic radii (RH) were measured for the rabFab′ conjugated with 20 kD and 40 kD PEG. Photon correlation spectroscopy was used to determine hydrodynamic radii (R_{H}), using Quasi-Elastic Light Scattering (QELS), with a single photon counting module with detection at a 99.0° (Wyatt Technology, Inc.). Raw data was obtained using Wyatt's proprietary Astra software, and molar mass and R_{H} constants were set in the Astra software using a rituximab standard. FIG. 12A is a graph depicting the correlation function used to measure RH using quasi elastic light scattering (QELS), and FIG. 12B is a graph of a representative QELS signal. The hydrodynamic volumes of the constructs were calculated assuming a spherical shape for the conjugated rabFab′ molecules. As summarized in Table 2, 20 kD PEGylation increased RH by about 2-fold to yield a value slightly larger than measured for the IgG format of the rabbit antibody. The RH increase with 40 kD PEGylation was about 2.7-fold relative to the unmodified rabFab′.

TABLE 2
Hydrodynamic Properties of Rabbit Constructs

| Construct | R_{H} (nm) | Calculated Volume (nm³) |
|---|---|---|
| Fab | 2.5 ± 0.2 | 65.5 |
| IgG | 4.9 ± 0.2 | 492.8 |
| 20KPEG-Fab′ | 5.2 ± 0.3 | 589.0 |
| 40KPEG-Fab′ | 6.9 ± 0.3 | 1376.1 |

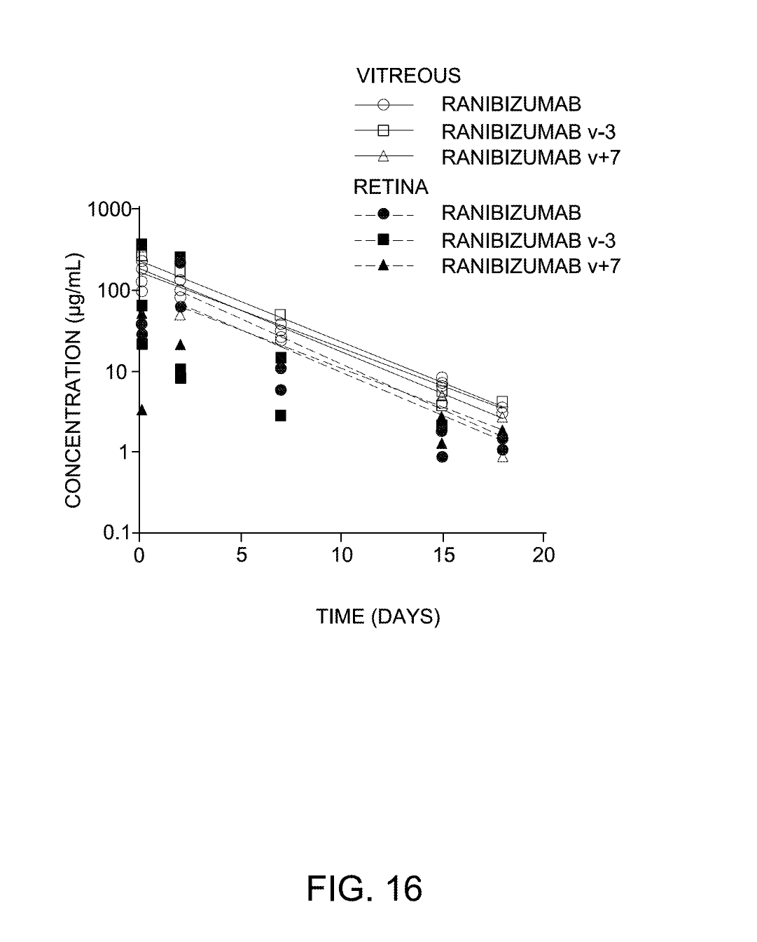

The time dependent vitreal concentration profiles observed for 20 kD and 40 kD PEGylated rabFab′ conjugates following 0.3 mg IVT dose in rabbits are shown in FIG. 16. As observed for rabFab′ in Ex. 1 above, no evidence was observed for an immune response against the PEGylated rabFab′ antibody conjugates. As summarized in Table 1 above, the vitreal half-life increased 1.5-fold for 20 kD PEGylated rabFab′ conjugate and approximately 2-fold for the 40 kD PEGylated conjugate.

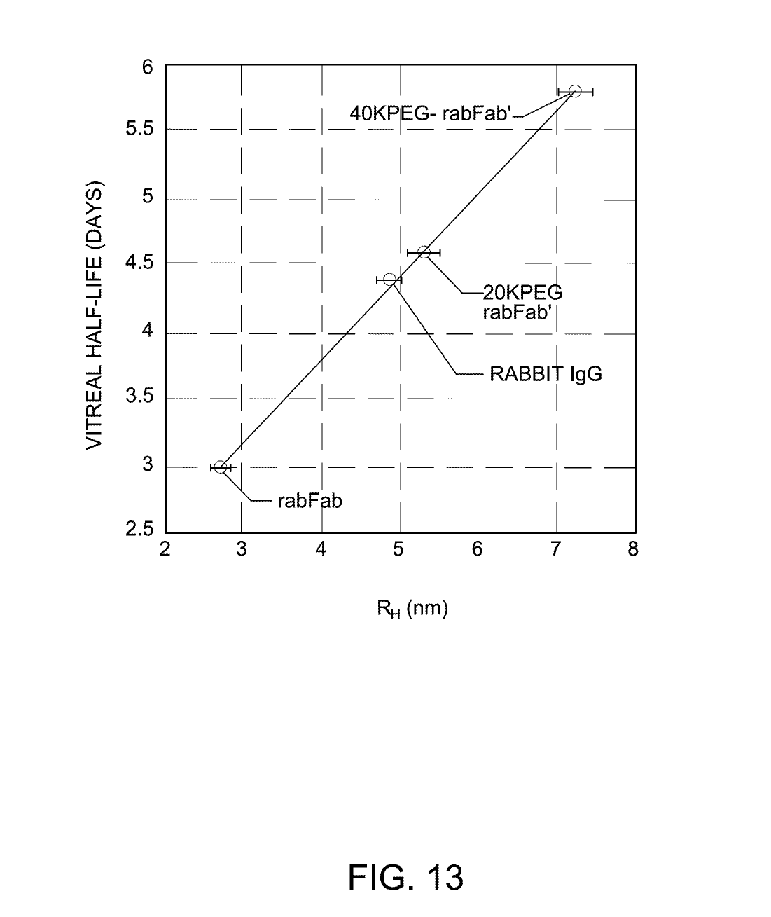

FIG. 13 is a graph summarizing the vitreal half-lives as a function of measured hydrodynamic radius. As illustrated in FIG. 13, the measured half-lives were determined to be directly proportional to the corresponding hydrodynamic radii (RH). Although the data set of the experiment was of limited range (RH values varied only over a 2.7-fold range), the vitreal half-lives showed a strong linear dependence on RH with a slope of 0.62 (R²=0.9995) within this RH range. By contrast, when the measured vitreal half-lives were plotted against the corresponding molecular weights, a relatively poor correlation (R²=0.53425) resulted, as illustrated in FIG. 2.

As an extension of these experiments, methods similar to those described above were used to evaluate the vitreal half-lives of rabFab conjugated to a high MW linear hyaluronic acid (HA). As illustrated in FIG. 1, the vitreal half-life of the high MW HA-conjugated form of rabFab was consistent with the linear correlation derived for the lower MW PEG conjugates described previously above.

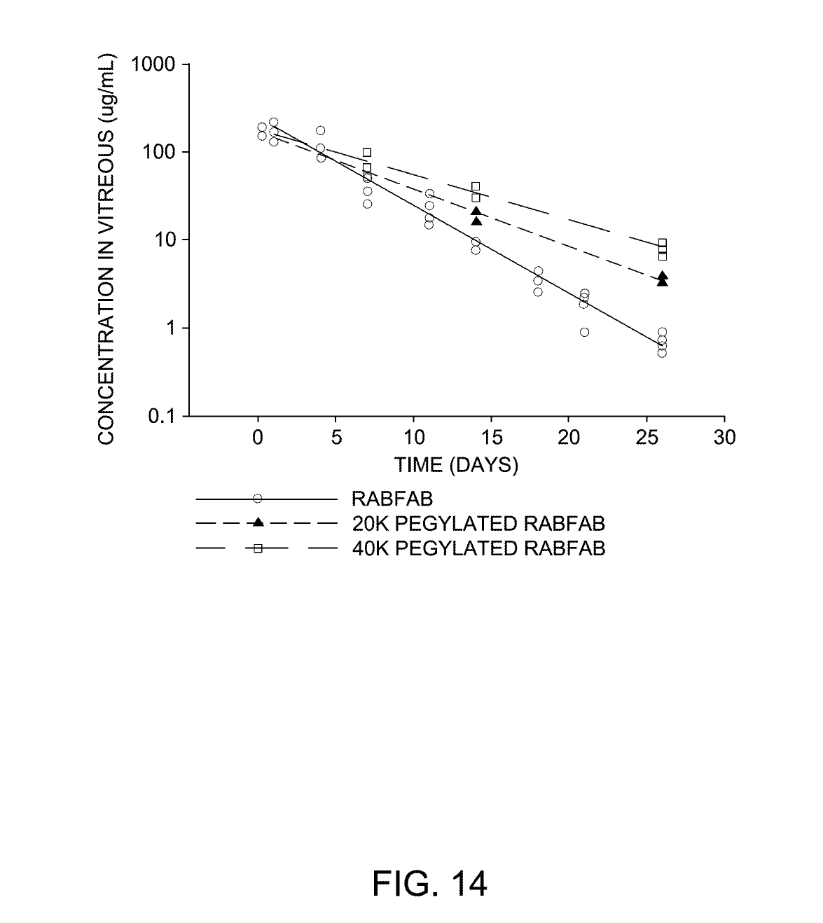

Referring again to Table 2 above, the half-life observed upon intravitreal injection of rabFab′ in NZW rabbits was consistent with that observed for humanized Fab fragments. Relative little variability in vitreous concentration levels was observed during clearance of the rabFab′ or either of the PEGylated conjugates after cessation of prolonged dosing (see FIG. 14), indicating an absence of an immune response against any of the tested compounds.

### Example 3: Effect of FcRn Binding on Vitreal Half-Life

To test the effect of FcRn binding on vitreal clearance kinetics, two mutated forms of IgG were assessed for vitreal half-life using the rabbit model and methods described in Ex. 1.

The influence of binding to the recycling FcRn receptor on vitreal half-life of IgG was tested with antibody mutations known to ablate FcRn-binding. For IgG antibodies in circulation, FcRn promotes long half-life by protecting IgG from catabolism upon non-specific pinocytosis (Roopenian & Akilesh (2007) Nat. Rev. Imm. 7:715). Substitutions H310A and H435Q in the Fc region of an antibody are associated with greatly reduced FcRn binding and fast systemic clearance (Kenanova et al. (2005) Cancer Res. 65:622). These substitutions were introduced into the Fc portion of the humanized 5B6 (anti-gD) antibody using oligonucleotide-directed mutagenesis as described in Ex. 4 below. Wild-type and H310A:H435Q variant anti-gD were expressed by transient expression in CHO cells and the secreted IgG antibody was purified by chromatography on Protein A-Sepharose followed by cation exchange chromatography on S-Sepharose. SPR measurements indicated the wild-type antibody bound to human FcRn with a K_{D} of 1.5 μM whereas no detectable binding at a concentration of 10 μM was observed for the H310A:H435Q (FcRn null) variant with both human and rabbit FcRn. The vitreal clearance kinetics of the wild-type and mutated antibodies were tested in rabbit pharmacokinetic experiments with intravitreal dosing as described in Ex. 1. Pharmacokinetics were also determined for antibodies administered by intravenous (IV) injection.

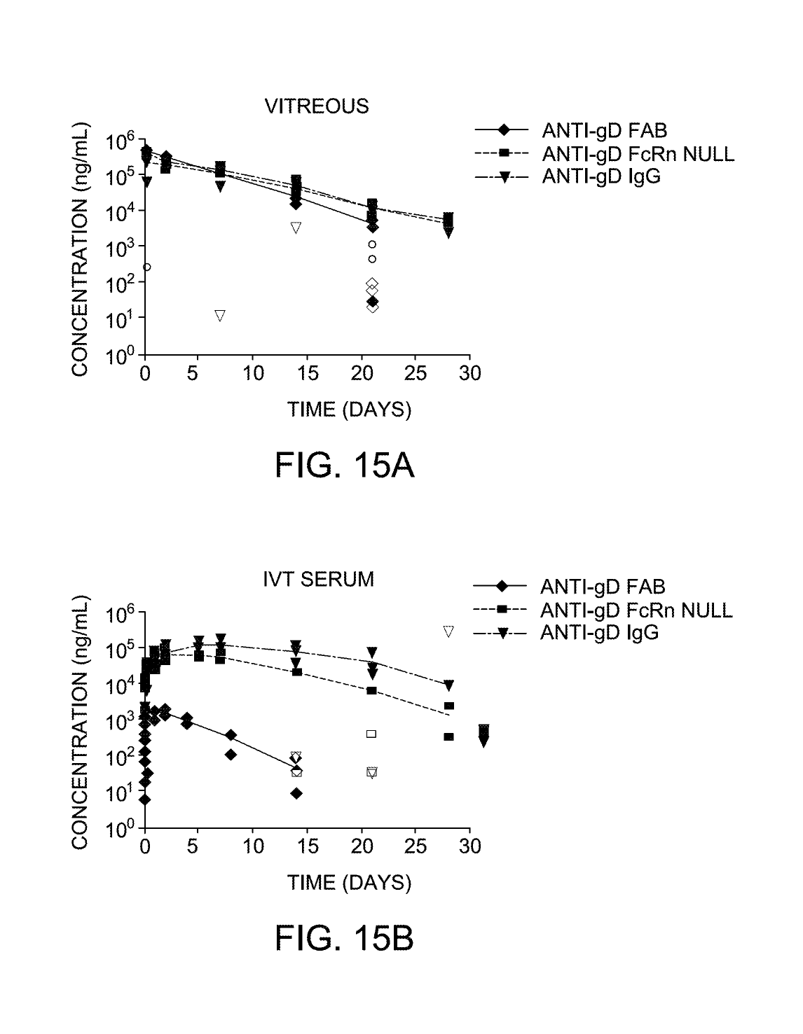

FIG. 15A is a graph summarizing the vitreal concentrations of the wild-type IgG, FcRn null IgG, and human Fab as a function of time post-intravitreal injection. FIG. 15B is a graph summarizing the serum concentrations of the wild-type IgG, FcRn null IgG, and human Fab as a function of time post-IV injection. As shown in FIG. 15A, the concentration profiles of the wild-type IgG and FcRn IgG were essentially identical, indicating that FcRn binding did not impact vitreal half-life of FcRn null IgG relative to wild-type IgG. The Fab vitreal concentrations decreased faster than the corresponding vitreal concentrations of the IgGs, which was consistent with a size dependence on the rate of vitreal clearance. Referring to FIG. 15B, the FcRn null IgG concentrations decreased more rapidly for the FcRn null IgG relative to the wild-type IgG in the serum, which was consistent with previously observed effects of FcRn binding to IgGs in the serum.

The results of this experiment indicated that binding to the FcRn receptor did not make a significant contribution to the vitreal half-life of IgG molecules.

### Example 4: Effect of Net Charge of Active Pharmaceutical Ingredient on Vitreal Half-Life

The effect of molecular charge variation on ocular pharmacokinetics was examined by producing designed molecular charge variants of ranibizumab. Since it was intended to use a VEGF-binding ELISA assay for detection of these variants in rabbit ocular tissues, a strategy to introduce amino acid changes with minimal impact on antigen binding was devised. The 3D structure previously determined for ranibizumab in complex with VEGF (Chen. J. Mol. Biol. 1999; 293:865) was examined to determine CDRs not in contact with the target antigen. Based on this examination, CDRs L1 and L2 were identified as regions of the ranibizumab molecule within which amino acid substitutions would be likely to have minimal impact on antigen binding. Positions within CDRs L1 and L2 known to tolerate the substitution of charged residues were selected from databases of human antibody sequences (e.g., Kabat et al., Sequences of Proteins of Immunological Interest, Fifth Edition, NIH Publication 91-3242, Bethesda Md. (1991), vols. 1-3.

The mutations were introduced by site-directed mutagenesis using the QuikChange® (Agilent) mutagenesis kit following the protocol supplied with the kit. Oligonucleotide primers specifying the required codon changes were synthesized and plasmids with the designed changes were identified and confirmed by DNA sequencing. For small scale expression and purification, DNA was transformed into the E. coli strain 64B4, and the transformed cells were grown overnight in low phosphate-containing media. Fab was purified from cell lysates prepared using PopCulture® (EMD Millipore) extraction buffer through chromatography on Protein G GraviTrap (GE Healthcare). For larger scale purification, cell paste from 10 L fermentation of the transformed cells was suspended in extraction buffer, homogenized using a microfluidizer, and the Fabs from the homogenized suspension were captured by immunoaffinity chromatography on Protein G—Sepharose and eluted with a low pH buffer. The low pH eluate was adjusted to pH 5 and further purified by cation exchange chromatography on an S-Sepharose column. Identities of the purified proteins were confirmed by mass spectroscopy and the pooled fractions were concentrated to about 10 mg/mL, and exchanged into PBS buffer, via diafiltration. Surface plasmon resonance (SPR) measurements on a Biacore® T200 instrument indicated that both the +7 and −3 molecular charge variants of ranibizumab retained high binding affinity to VEGF.

Unmodified ranibizumab, as well as the +7 and −3 molecular charge variants of ranibizumab were administered to the rabbits using the methods of Ex. 1. The unmodified ranibizumab and the molecular charge variants were administered via a single bilateral intravitreal injection to rabbits, and the rabbits were observed for up to 27 days post-injection. Vitreal and retinal samples were obtained using methods similar to those described in Ex. 1.

Antibody Fab in retinal tissue was extracted by homogenization in 50 mM Tris-HCl pH 8.0, 1 M NaCl. Vitreous and retinal concentrations of test articles was determined using a VEGF-binding ELISA assay similar to the ELISA assay described in Ex. 1. Values below the lower limit of quantitation (LLOQ) were not used in pharmacokinetic analysis or for graphical or summary purposes. Pharmacokinetic parameters were determined by non-compartmental analysis with nominal time and dose (Phoenix WinNonlin, Pharsight Corp, Mountain View, Calif.).

Concentration time profiles for the unmodified ranibizumab and the molecular charge variants are shown in FIG. 16, and the pharmacokinetics parameters calculated from the non-compartmental analysis are summarized in Table 3. These results indicated that charge variation in ranibizumab did not have a significant effect on ocular pharmacokinetics.

TABLE 3
Vitreal Kinetics of Charged Ranibizumab Variants

| | AUCₐₗₗ | ½-life | CL | V_{d} | Cₘₐₓ |
|---|---|---|---|---|---|
| Molecule | (day * μg/mL) | (days) | (mL/day) | (mL) | (μg/mL) |
| Ranibizumab | 822 | 3.3 | 0.36 | 1.4 | 184 |
| Ranibizumab v + | 1090 | 3.0 | 0.27 | 1.0 | 246 |
| 7 | | | | | |
| Ranibizumab v − | 865 | 3.0 | 0.34 | 1.2 | 219 |
| 3 | | | | | |

The results of this experiment demonstrated that variation in molecular charge between test molecules did not make a significant contribution to vitreal half-life.

### Example 5: Comparison of Estimated and Measured Vitreal Half-Lives

To evaluate the accuracy of the predetermined vitreal half-life-RH relation described herein previously, the following experiment was conducted.

Previously published values of vitreal half-life were predicted using the published values of hydrodynamic radius in the predetermined vitreal half-life-RH relation expressed as Eqn. (2) described herein above. The published vitreal half-lives were compared to the corresponding predicted vitreal half-lives to assess the accuracy of the predetermined vitreal half-life-RH relation.

The results of this comparison are summarized in Table 4 below. In general, the predicted vitreal half-lives compared favorably with the corresponding literature values, although there appeared to be a slight divergence between predicted and literature vitreal half-lives for the 18 kD HA molecule.

TABLE 4
Comparison of Published and Predicted Vitreal Kinetics of Compounds

| | | Predicted | Literature value for |
|---|---|---|---|
| | | vitreal half- | vitreal half-life |
| Molecule | Rh (nm) | life (days) | in rabbit (days) |
| 18 kD HA | 9 | 6.8 | 4 |
| 500 kD HA | 45 | 28 | 30 |
| albumin | 3.5 | 3.6 | 4 |
| Aflibercept | 6 | 5 | 4.6 |

### Example 6: Hyaluronic Acid-Anti-VEGF Conjugate Preparation

Hyaluronic acid (HA)-anti-VEGF conjugate may be prepared as described by WO 2011/066417. Briefly, hyaluronic acid (10 mg, 6.25 nmol; Sigma-Aldrich, St. Louis, Mo.) may be dissolved in 1 mL perphosphate buffer solution (pH 7.4). EDC (N-(3-dimethyl-aminopropyl)-N′-ethylcarbodiimide hydrochloride, 120 mg, 625 nmol; Sigma-Aldrich, St. Louis, Mo.), sulfo-NHS (N-hydroxysulfosuccinimide sodium salt, 217 mg, 1 mmole; Sigma-Aldrich, St. Louis, Mo.), and 4-DMAP (4-(dimethylamino)pyridine, 10 mg; Sigma-Aldrich, St. Louis, Mo.) may be added as solids to the HA solution and allowed to dissolve and react overnight. Anti-human VEGF monoclonal antibody (0.5 mg; R&D Systems Inc., Minneapolis, Minn.) may be added to the activated hyaluronic acid solution and stirred at 4° C. overnight. The solution may be dialyzed (MW cut-off 300 kDa) using a spin dialysis against PBS for 16 hrs. with 4 changes of PBS solution.

### Example 7: Hyaluronic Acid anti-Flt1 Conjugate Preparation

Hyaluronic acid anti-Flt1 conjugate may be prepared as described by Oh et al., Biomaterials (2009) Vol. 30, pp. 6029-6034.

Tetra-n-butyl ammonium hyaluronate may be prepared as described by Oh et al., Bioconjugate Chem., (2008) Vol. 19, pp 2401-2408. Dowex® 50WX8-400 ion-exchange resin (12.5 g; Sigma-Aldrich, St. Louis, Mo.) may be washed with water, and then excess 1.5 M tetra-n-butyl ammonium hydroxide may be added to the Dowex resin and mixed for 30 min. The resulting resin may be filtered to remove the supernatant. Sodium hyaluronate (MW-100 kDa, 1 g; Shiseido Co., Tokyo, Japan) may be dissolved in 100 mL of water, and poured into the prepared Dowex resin (10 g). After mixing for 3 h, the supernatant may be filtered through a 0.45 μm filter to remove the resin and provide tetra-it-butyl ammonium hyaluronate as a clear solution, which may be lyophylized.

Anti-Flt1 peptide (amino acid sequences GNQWFL KGNQWFI, or GGNQWFI; Peptron Co., Daejeon, Korea) and tetra-n-butyl ammonium hyaluronate may each be dissolved in DMSO, separately, after which BOP (benzotriazol-1-oxy-tris(dimethylamino)phosphonium hexafluorophosphate; Sigma-Aldrich, St. Louis, Mo.) may be added to the tetra-n-butyl ammonium hyaluronate and mixed for 30 min. The tetra-n-butyl ammonium hyaluronate solution may then be mixed with the anti-Flt1 peptide and DIPEA (N,N-diisopropyl ethyiamine; Sigma-Aldrich, St. Louis, Mo.) dissolved in DMSO. After reaction at 37° C. for a day, 1 M NaCl aqueous solution may be added with a volume ratio of 1/1. The pH of the solution may be reduced to 3.0 by addition of 1M HCl and then raised to 7.0 by addition of 1M NaOH. The resulting product may be dialyzed against excess mixture of 0.3 M NaCl solution, 25% ethanol, and water, and lyophilized.

### Example 8: Preparation of PEGylated FAB Conjugates

Fabs, such as ranibizumab or the Fab portion of bevacizumab, may be prepared as described in “Antibody Protocols and Methods,” Springer 2012, Proetzel, Ed. (“PEGylation of Antibody Fragments for Half-life Extension,”, Jesevar et al., Chapter 15, pp. 233-246). Briefly, the desired Fab (10 mg) may be dissolved in sodium phosphate buffer to about 2.5 mg/mL. A sodium phosphate buffer solution of tris-(2-carboxyethyl) phosphine hydrochloride (TCEP) may be added until TCEP concentration is about 0.1 uL, and the mixture may be incubated at room temperature with shaking for 90 min, after which excess TCEP may be removed via spin dialysis against sodium phosphate buffer. The Fab solution may be incubated for 24 hr. to allow reconstitution of interchain disulfide bridges, 400 μL of PEG maleimide solution (SUNBRIGHT ME-200MA, 300 mg in 600 μL of sodium phosphate buffer, NOF Corporation) may be added, and the resulting mixture may be incubated overnight. The mixture may then be diluted with acetic acid buffer, filtered, and the filtrate may be passed through a TSK-GEL SP-5PW resin column (Tosoh, Inc.) to provide the PEGylated Fab.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

1. A method for identifying a therapeutic agent-polymer conjugate having a preselected vitreal half-life, the method comprising:
a) determining a hydrodynamic radius (RH) of the therapeutic agent-polymer conjugate;
b) transforming the RH to a predicted vitreal half-life of the therapeutic agent-polymer conjugate according to a predetermined vitreal half-life-RH relation; and
c) assessing whether the predicted vitreal half-life is greater than or equal to the preselected vitreal half-life.

2. The method of claim 1, wherein the predetermined vitreal half-life-RH relation is obtained empirically by correlating a plurality of vitreal half-lives measured for a plurality of therapeutic agent-polymer conjugates with a plurality of measured hydrodynamic radii (RH) measured for the plurality of therapeutic agent-polymer conjugates.

3. The method of claim 2, wherein the predetermined vitreal half-life-RH relation is obtained empirically using a linear regression method.

4. The method of claim 3, wherein the predetermined vitreal half-life-RH relation is expressed as Eqn. (1):
Y=(1.53±0.005)+(0.588±0.005)X Eqn. (1)
wherein:
Y is the predicted vitreal half-life in days;
X is the RH in nm; and
the predetermined vitreal half-life-RH relation expressed by Eqn. (1) further comprises a correlation coefficient (R²) of greater than or equal to about 0.9.

5. The method of claim 3, wherein the predetermined vitreal half-life-RH relation is expressed as Eqn. (2):
Y=1.5322+0.58834X Eqn. (2)
wherein:
Y is the predicted vitreal half-life in days;
X is the RH in nm; and
the predetermined vitreal half-life-RH relation expressed by Eqn. (2) further comprises a correlation coefficient (R²) of greater than or equal to about 0.97434.

6. The method of claim 1, wherein the therapeutic agent-polymer conjugate comprises a polymer moiety selected from the group consisting of polyethylene glycol (PEG), hyaluronic acid, hydroxyethyl starch, heparosan, phosphorylcholine polymer, and 2-polyalkyloxazoline.

7. The method of claim 6, wherein the polymer moiety is polyethylene glycol (PEG).

8. The method of claim 7, wherein the PEG is branched.

9. The method of claim 8, wherein the branched PEG comprises a multi-arm PEG selected from a 2-armed PEG, a 3-armed PEG, a 4-armed PEG, a 5-armed PEG, a 6-armed PEG, a 7-armed PEG, an 8-armed PEG, a 9-armed PEG, a 10-armed PEG, a 11-armed PEG, and a 12-armed PEG.

10. The method of claim 9, wherein the multi-arm PEG is selected from a 4-armed PEG, a 6-armed PEG, and an 8-armed PEG.

11. The method of claim 1, wherein the therapeutic agent is an antibody or a fragment thereof.

12. The method of claim 11, wherein the antibody fragment is a Fab fragment.

13. The method of claim 6, wherein the molecular weight of a polymer moiety of the therapeutic agent-polymer conjugate is greater than or equal to about 1000 Daltons.

14. The method of claim 6, wherein the polymer moiety of the therapeutic agent-polymer conjugate has an average molecular weight ranging from about 1000 Daltons to about 500000 Daltons.

15. The method of claim 1, wherein the hydrodynamic radius of the therapeutic agent-polymer conjugate is greater than or equal to about 1 nm.

16. The method of claim 1, wherein the hydrodynamic radius of the therapeutic agent-polymer conjugate ranges from about 1 nm to about 50 nm.

17. The method of claim 1, wherein the hydrodynamic radius of the therapeutic agent-polymer conjugate ranges from about 1 nm to about 25 nm.

18. The method of claim 1, wherein the hydrodynamic radius of the therapeutic agent-polymer conjugate ranges from about 1 nm to about 15 nm.

19. The method of claim 1, wherein the hydrodynamic radius of the therapeutic agent-polymer conjugate ranges from about 1 nm to about 10 nm.

20. The method of claim 1, wherein the hydrodynamic radius of the therapeutic agent-polymer conjugate ranges from about 2 nm to about 8 nm.

21. The method of claim 1, further comprising:
d) modifying the polymer moiety of the therapeutic agent-polymer conjugate to increase the RH if the predicted vitreal half-life is less than the preselected vitreal half-life, and repeating a)-c) until the predicted vitreal half-life of the conjugate is greater than or equal to the preselected vitreal half-life; and
e) selecting the therapeutic agent-polymer conjugate from d) wherein the predicted vitreal half-life of the conjugate is greater than or equal to the preselected vitreal half-life.

22. The method of claim 21, further comprising:
f) determining an in vivo vitreal half-life of the therapeutic agent-polymer conjugate from c) using an animal model.

23. A method of selecting a therapeutic agent-polymer conjugate for use in an ocular therapy, the therapeutic agent-polymer conjugate having a predicted vitreal half-life that is greater than or equal to a preselected vitreal half-life, the method comprising:
a) preparing a plurality of candidate therapeutic agent-polymer conjugates, wherein each candidate therapeutic agent-polymer conjugate of the plurality comprises the therapeutic agent and a polymer moiety, each polymer moiety comprising a different composition than each other polymer moiety in the plurality;
b) determining a hydrodynamic radius (RH) for each therapeutic agent-polymer conjugate of the plurality;
c) transforming each RH to a predicted vitreal half-life for each therapeutic agent-polymer conjugate of the plurality according to a predetermined vitreal half-life-RH relation;
d) assessing whether each predicted vitreal half-life is greater than or equal to the preselected vitreal half-life; and
e) selecting one candidate therapeutic agent-polymer conjugate from among the plurality of candidate therapeutic agent-polymer conjugates, wherein the selected candidate therapeutic agent-polymer conjugate is characterized by a predicted vitreal half-life that is greater than or equal to the preselected vitreal half-life for the ocular treatment.

24. The method of claim 23, further comprising preparing the selected candidate therapeutic agent-polymer conjugate in a quantity sufficient to provide a dosage to at least one patient.

25. The method of claim 23, further comprising packaging at least one dosage in a storage device suitable for administration of the dosage to a patient.

26. The method of claim 25, wherein the packaging comprises a pre-filled syringe configured for injection into the eye of a patient.

27. The method of claim 25, wherein the packaging comprises an ampoule/vial configured to permit withdrawal of at least one of the dosages via a syringe.

28. A method for identifying a therapeutic agent-polymer conjugate having a preselected vitreal half-life, the method implemented by a computing device including at least one processor in communication with a memory, the method comprising:
a) receiving, by the computing device, a hydrodynamic radius (RH) of the therapeutic agent-polymer conjugate;
b) transforming, by the computing device, the RH to a predicted vitreal half-life of the therapeutic agent-polymer conjugate according to a predetermined vitreal half-life-RH relation;
c) assessing whether the predicted vitreal half-life is greater than or equal to the preselected vitreal half-life; and
d) displaying, by the computing device, on a user interface of the computing device, the predicted vitreal half-life.

29. The method of claim 28, wherein the RH of the therapeutic agent-polymer conjugate is selected from the group consisting of an RH measured from a sample of the therapeutic agent-polymer conjugate; an RH estimated from a chemical structure of the therapeutic agent-polymer conjugate; and a published RH value for the therapeutic agent-polymer conjugate.

30. The method of claim 28, wherein the RH is measured using a method selected from: quasi elastic light scattering (QELS), fluorescence correlation spectroscopy (FCS), pulse field NMR, and UV area imaging.

31. The method of claim 28, wherein the RH is measured using quasi elastic light scattering (QELS).

32. The method of claim 28, wherein the predetermined vitreal half-life-RH relation is obtained empirically by correlating a plurality of vitreal half-lives measured for a plurality of therapeutic agent-polymer conjugates with a plurality of measured hydrodynamic radii (RH) measured for the plurality of therapeutic agent-polymer conjugates.

33. The method of claim 28, wherein the predetermined vitreal half-life-RH relation is obtained empirically using a linear regression method.

34. The method of claim 33, wherein the predetermined vitreal half-life-RH relation is expressed as Eqn. (1):
Y=(1.53±0.005)+(0.588±0.005)X Eqn. (1)
wherein:
Y is the predicted vitreal half-life in days;
X is the RH in nm; and
the predetermined vitreal half-life-RH relation expressed by Eqn. (1) further comprises a correlation coefficient (R²) of greater than or equal to about 0.9.

35. The method of claim 33, wherein the predetermined vitreal half-life-RH relation is expressed as Eqn. (2):
Y=1.5322+0.58834X Eqn. (2)
wherein:
Y is the predicted vitreal half-life in days;
X is the RH in nm; and
the predetermined vitreal half-life-RH relation expressed by Eqn. (2) further comprises a correlation coefficient (R²) of greater than or equal to about 0.97434.

36. The method of claim 28, further comprising:
d) displaying, by the computing device, on a user interface of the computing device, the therapeutic agent-polymer conjugate comprising the therapeutic agent and the polymer moiety, and the predicted vitreal half-life; and
e) modifying the polymer moiety of the therapeutic agent-polymer conjugate to increase the RH if the predicted vitreal half-life is less than the preselected vitreal half-life, and repeating a)-d) until the predicted vitreal half-life of the conjugate is greater than or equal to the preselected vitreal half-life.

37. A computing device comprising at least one processor in communication with a memory, the at least one processor programmed to:
a) receive a hydrodynamic radius (RH) of the therapeutic agent-polymer conjugate;
b) transform the RH to a predicted vitreal half-life of the therapeutic agent-polymer conjugate according to a predetermined vitreal half-life-RH relation;
c) assess whether the predicted vitreal half-life is at least the preselected vitreal half-life; and
d) display, on a user interface of the computing device, the therapeutic agent-polymer conjugate comprising the therapeutic agent and the modified polymer moiety, and the predicted vitreal half-life.

38. The computing device of claim 37, wherein the at least one processor is further programmed to:
e) modify the polymer moiety of the therapeutic agent-polymer conjugate to increase the RH if the predicted vitreal half-life is less than the preselected vitreal half-life, and repeat a)-d) until the predicted vitreal half-life of the conjugate is greater than or equal to the preselected vitreal half-life.

39. The computing device of claim 38, wherein the polymer moiety is modified by the computing device.

40. A computer-readable storage medium having computer-executable instructions embodied thereon, wherein when executed by a computing device including at least one processor in communication with a memory, the computer-executable instructions cause the computing device to:
a) receive a hydrodynamic radius (RH) of the therapeutic agent-polymer conjugate;
b) transform the RH to a predicted vitreal half-life of the therapeutic agent-polymer conjugate according to a predetermined vitreal half-life-RH relation;
c) assess whether the predicted vitreal half-life is greater than or equal to the preselected vitreal half-life; and
d) display, on a user interface of the computing device, the therapeutic agent-polymer conjugate comprising the therapeutic agent and the modified polymer moiety, and the predicted vitreal half-life.

41. The computer-readable storage medium of claim 40, wherein the computer-executable instructions further cause the computing device to:
e) modify the polymer moiety of the therapeutic agent-polymer conjugate to increase the RH if the predicted vitreal half-life is less than the preselected vitreal half-life, and repeat a)-d) until the predicted vitreal half-life of the conjugate is greater than or equal to the preselected vitreal half-life.

42. The computer-readable storage medium of claim 40, wherein the computer-executable instructions further cause the computing device to modify the polymer moiety.

43. A system for identifying a therapeutic agent-polymer conjugate having a preselected vitreal half-life using a computing device comprising at least one processor in communication with a memory, the memory comprising a plurality of modules, each module comprising instructions configured to execute using the at least one processor, the plurality of modules comprising:
a) a first module to receive a hydrodynamic radius (RH) of the therapeutic agent-polymer conjugate;
b) a second module to transform the RH to a predicted vitreal half-life of the therapeutic agent-polymer conjugate according to a predetermined vitreal half-life-RH relation;
c) a third module to assess whether the predicted vitreal half-life is at least the preselected vitreal half-life; and
d) a fourth module to display, on a user interface of the computing device, the therapeutic agent-polymer conjugate comprising the therapeutic agent and the modified polymer moiety, and the predicted vitreal half-life.

44. The system of claim 43, wherein the plurality of modules further comprise a fifth module to modify the polymer moiety of the therapeutic agent-polymer conjugate to increase the RH if the predicted vitreal half-life is less than the preselected vitreal half-life, and to re-execute the instructions of the first, second, third, and fourth modules until the predicted vitreal half-life of the conjugate is greater than or equal to the preselected vitreal half-life.
